(19) United States
(12) Patent Application Publication
(10) Pub. No.: US 2026/0234310 A1
(43) Pub. Date: Aug. 13, 2026

(54) MODIFIED CROSSLINKED POLYMERS

(71) Applicant: LifeSprout, Inc., Baltimore, MD (US)

(72) Inventors: Russell Andrew Martin, Baltimore, MD (US); Xuesong Jiang, Lutherville-Timonium, MD (US)

(73) Assignee: LifeSprout, Inc., Baltimore, MD (US)

(21) Appl. No.: 19/156,739

(22) PCT Filed: Dec. 27, 2023
(86) PCT No.: PCT/US2023/086094

§ 371 Date: Aug. 14, 2025

Related U.S. Application Data
(60) Provisional application No. 63/485,164, filed on Feb. 15, 2023.

(51) Int. Cl. C08F 251/00; A61K 8/04; A61K 8/65; A61K 8/73; A61Q 19/00; C08K 5/36

(52) U.S. Cl. CPC C08F 251/00; A61K 8/042; A61K 8/65; A61K 8/73; A61Q 19/00; C08K 5/36; A61K 2800/54; A61K 2800/805; A61K 2800/91

(57) ABSTRACT

Disclosed herein are methods and compositions directed to crosslinked hydrogel forms produced by chemically quenching residual pendant groups. Aspects of the present invention include favorable biocompatibility profiles for DVS-crosslinked products, and removal of residual unreacted crosslinking agent left in a medical device thereby reducing or minimizing toxicity based on pendant groups.

### RELATED APPLICATIONS

The present application claims the benefit of priority of U.S. provisional application No. 63/485,164; filed Feb. 15, 2023, which is incorporated by reference herein in its entirety.

### TECHNICAL FIELD

The disclosure relates generally to crosslinked polymer materials and methods for minimizing toxicity of unreacted or partially reacted crosslinking agents. The disclosure also relates to compositions and methods directed to hydrogel materials crosslinked with divinyl sulfone comprising reduced numbers of pendant groups.

### BACKGROUND

Hydrogels are popular scaffold matrices for tissue restoration in the field of soft tissue repair because of their biomimetic features close to the tissue microenvironment and their ability to be tailored for different adipose tissue needs such as volume and porosity (Young, D. A., et al., Acta biomaterialia, 2011. 7(3): p. 1040-1049; Varma, D. M., et al., Acta Biomaterialia, 2014. 10(12): p. 4996-5004).

Among various hydrogel products, hyaluronic acid (HA) has been widely used as the base material for fillers and foams for soft tissue augmentation because of its biocompatibility as an endogenous polysaccharide, its moderate biodegradability, and similar physical properties to soft tissues like adipose tissue, fibrous tissue, and nerves. (Tezel, A. and G. H. Fredrickson, The science of hyaluronic acid dermal fillers, Journal of Cosmetic and Laser Therapy, 2008. 10 (1): p. 35-42; Wang, F., et al., Archives of Dermatology, 2007. 143 (2): p. 155-163).

Crosslinked hydrogels (made from hyaluronic acid, carboxy methyl cellulose, collagen or equivalents) have widespread use in the medical field, in wound care, aesthetics, and other therapeutic indications. Divinyl sulfone (DVS) and 1,4-butanediol diglycidyl ether (BDDE) diepoxyoctane (DEO), biscarbodiimide (BCDI), and polyethylene glycol diglycidyl ether (PEG-DE) are examples of crosslinking reagents for forming network hydrogels such as hyaluronic acid gels. However, BDDE, DEO, BCDI, PEG-DE, and DVS crosslinkers are extremely toxic. The toxicity of these crosslinkers comes from the ability of the crosslinker functional groups to react with constituents in the body after injection. (See: J. D. West, et al. “Enhanced toxicity of the protein cross-linkers divinyl sulfone and diethyl acetylenedicarboxylate in comparison to related monofunctional electrophiles.” Chemical research in toxicology 24.9 (2011): 1457-1459.)

BDDE has become the dominant crosslinker in the market over DVS. BDDE-crosslinked gels have been replacing DVS-crosslinked gels in part due to the safety profile of BDDE vs DVS. DVS is significantly more toxic than BDDE. (See: J. Faivre, et al. (2021) “Crosslinking hyaluronic acid soft-tissue fillers: current status and perspectives from an industrial point of view, Expert Review of Medical Devices,” 18:12, 1175-1187.) Unreacted BDDE residuals may be further reduced through hydrolysis during autoclave treatment, at a much greater rate than any hydrolysis of residual vinyl sulfone groups. Soft tissue fillers that are crosslinked with DVS (divinyl sulfone): Hylaform™ and Captique,™ are no-longer marketed in the USA, at least in part due to safety concerns.

A key driver of obtaining a favorable biocompatibility profile for DVS-crosslinked products, therefore, is to reduce or eliminate the amount of residual unreacted crosslinker left in the final device. A focus of the product development phase and associated regulatory scrutiny is on the purification processes to remove unreacted crosslinking agents. Further methods and compositions for reducing or minimizing toxicity are needed.

### SUMMARY OF THE DISCLOSURE

The disclosure provides a method of manufacturing a crosslinked polymer comprising steps of:
(A) reacting a polymer material and crosslinking agent, wherein the crosslinking agent comprises two or more vinyl sulfone groups,
whereby the polymer is crosslinked by the vinyl sulfone groups, and
whereby the crosslinked polymer comprises a pendant vinyl sulfone group; and
(B) reacting the pendant vinyl sulfone group with a quenching agent;
whereby the pendant vinyl sulfone group is quenched.

The disclosure provides a method of manufacturing a hydrogel network comprising steps of:
(A) reacting a hydroxylated polymer and a crosslinking agent, wherein the crosslinking agent comprises two or more vinyl sulfone groups,
whereby the hydroxylated polymer is crosslinked by the vinyl sulfone groups,
whereby a crosslinked hydrogel material is formed,
wherein the crosslinked hydrogel material comprises a pendant vinyl sulfone groups; and
(B) reacting the pendant vinyl sulfone group with a quenching agent
whereby the hydrogel network is formed.

The disclosure provides a method of manufacturing a hydrogel comprising steps of:
(A) reacting a hyaluronic acid material and a crosslinking agent, wherein the crosslinking agent comprises two or more vinyl sulfone groups,
whereby the hyaluronic acid is covalently crosslinked by the vinyl sulfone groups,
whereby a crosslinked hyaluronic acid is formed,
wherein the crosslinked hyaluronic acid comprises a pendant vinyl sulfone groups; and
(B) reacting the pendant vinyl sulfone group with a quenching agent
whereby the hydrogel is formed.

The disclosure provides a method of manufacturing a hydrogel comprising steps of:
(A) reacting a polysaccharide and a crosslinking agent, wherein the crosslinking agent comprises two or more vinyl sulfone groups,
whereby the polysaccharide acid is covalently crosslinked by the vinyl sulfone groups,
whereby a crosslinked hydrogel is formed,
wherein the crosslinked hydrogel comprises one or more pendant vinyl sulfone groups; and
(B) reacting the pendant vinyl sulfone group(s) with a quenching agent
whereby the hydrogel is formed.

The disclosure provides a method of manufacturing a crosslinked polymer comprising steps of:
(A) reacting a polymer material and a divinyl sulfone,
whereby the polymer is crosslinked by the divinyl sulfone, and
whereby the crosslinked polymer comprises a pendant vinyl sulfone groups; and
(B) reacting the pendant vinyl sulfone group with a quenching agent;
whereby the pendant vinyl sulfone group is quenched.

The disclosure provides a method of manufacturing a hydrogel network comprising steps of:
(A) reacting a hydroxylated polymer and a divinyl sulfone,
whereby the hydroxylated polymer is crosslinked by the divinyl sulfone,
whereby a crosslinked hydrogel is formed,
wherein the crosslinked hydrogel comprises a pendant vinyl sulfone group; and
(B) reacting the pendant vinyl sulfone group with a quenching agent
whereby the hydrogel network is formed.

The disclosure provides a method of manufacturing a hydrogel comprising steps of:
(A) reacting a hyaluronic acid material and divinyl sulfone,
whereby the hyaluronic acid is covalently crosslinked by the divinyl sulfone,
whereby a crosslinked hyaluronic acid is formed,
wherein the crosslinked hyaluronic acid comprises a pendant vinyl sulfone groups; and
(B) reacting the pendant vinyl sulfone group with a quenching agent
whereby the hydrogel is formed.

The disclosure provides a hydrogel product by the process comprising the steps of:
(A) reacting a hyaluronic acid material and a crosslinking agent, wherein the crosslinking agent comprises two or more vinyl sulfone groups,
whereby the hyaluronic acid is covalently crosslinked by the vinyl sulfone groups,
wherein the hydrogel material comprising a vinyl sulfone pendant group; and
(B) reacting the vinyl sulfone pendant groups with an quenching agent;
thereby forming the hydrogel.

The disclosure provides a hydrogel product by the process comprising the steps of:
(A) reacting a hyaluronic acid material and divinyl sulfone,
   whereby the hyaluronic acid is covalently crosslinked by the divinyl sulfone,
   wherein the hydrogel material comprises vinyl sulfone pendant groups; and
(B) reacting the vinyl sulfone pendant groups with a quenching agent, thereby forming the hydrogel,
   wherein the hydrogel comprises a residual vinyl sulfone concentration of at most about 2.0 micromoles per mL of hydrogel.

The disclosure provides a hydrogel product by the process comprising the steps of:
(A) reacting a hyaluronic acid material and divinyl sulfone,
   whereby the hyaluronic acid is covalently crosslinked by the divinyl sulfone,
   wherein the hydrogel material comprises vinyl sulfone pendant groups; and
(B) reacting the vinyl sulfone pendant groups with a quenching agent, thereby forming the hydrogel,
   wherein the hydrogel comprises a residual vinyl sulfone concentration of at most about 0.15 micromoles per mg of hydrogel-former-component (such as HA) in the hydrogel.

The disclosure provides a hydrogel composition comprising:
a hyaluronic acid network comprising a plurality of hyaluronic acid chains,
a sulfonyl bis-ethyl crosslink between hyaluronic acid chains,
a pendant group
a quenching agent wherein the quenching agent is covalently bonded to the pendent group.

The disclosure provides a hydrogel composition comprising:

a hyaluronic acid network comprising a plurality of hyaluronic acid chains,
a sulfonyl bis-ethyl crosslink between hyaluronic acid chains,
a pendant group,
a quenching agent wherein the quenching agent is covalently bonded to the pendent group.

In one aspect, the present disclosure is directed to compositions and methods for making DVS-crosslinked polymers safer or more biocompatible.

Disclosed herein are methods and compositions for modifying a hydrogel material that has been crosslinked using a divinyl sulfone crosslinking agent.

Disclosed herein are methods and compositions for modifying a hydrogel material that has been crosslinked using a crosslinking agent that comprises a vinyl sulfone.

Disclosed herein are methods and compositions for modifying a hydrogel material that has been crosslinked using a divinyl sulfone crosslinking agent.

Disclosed herein are methods and compositions for modifying a polymer that has been crosslinked using a crosslinking agent that comprises a vinyl sulfone.

Disclosed herein are methods and compositions for modifying a polymer that has been crosslinked using a divinyl sulfone crosslinking agent.

Disclosed herein are methods and compositions for modifying a polysaccharide that has been crosslinked using a crosslinking agent that comprises a vinyl sulfone.

Disclosed herein are methods and compositions for modifying a polysaccharide that has been crosslinked using a divinyl sulfone crosslinking agent.

Disclosed herein are methods and compositions for producing a hydrogel material comprising a polysaccharide and a divinyl sulfone crosslinking agent.

Disclosed herein are methods and compositions for producing a hydrogel material comprising a hyaluronic acid (HA), and a divinyl sulfone crosslinking agent.

Disclosed herein are compositions comprising Hyaluronic acid-based gels with amino-acid-substituted vinyl sulfone pendant groups.

Disclosed herein are compositions comprising Hyaluronic acid-based gels with alternatively-substituted vinyl sulfone pendant groups (such as cysteamine, ethyl diamine, etc.).

Disclosed herein are methods for improving a DVS-crosslinked gel with lowered residual pendant vinyl sulfone groups for enhanced biocompatibility.

Disclosed herein are methods for reducing the toxicity of a DVS-crosslinked gel with residual pendant vinyl sulfone groups.

Disclosed herein are methods for quenching a crosslinking reaction of a polymer with DVS. Disclosed herein are methods for quenching a crosslinking reaction of a sugar polymer with DVS.

In various aspects, this disclosure relates a novel form of crosslinked Hyaluronic acid gel with an enhanced safety profile due to a chemical quenching of residual and pendant crosslinking groups. The modified structure additionally provides an additional means of directing cellular responses and device properties.

In a various aspects, a quenching agent is added to the reactant mixture at the end of gel crosslinking reaction, but before the acid quenching. In a various aspects, the quenching agent comprises a nucleophile, or Lewis-base. In a various aspects, the quenching agent comprises a nucleophile selected from the group consisting of a primary amine group and a primary thiol group.

In a various non limiting examples, a quenching agent bearing a primary amine or a primary thiol group (e.g., lysine, glycine, cysteine, or equivalent) is added to the reactant mixture at the end of gel crosslinking reaction, but before the acid quenching.

In various embodiments, the quenching agent is allowed time to diffuse and react with residual unreacted vinyl sulfone groups, prior to acid-quenching the reaction. In various embodiments, the quenching reaction time ranges from at least about 1 minute to about 1 hour or ranges from about 5 minutes to about 15 minutes at room temperature or above; or up to several hours at temperatures below about 25° C. This freely soluble, small molecule bearing a primary amine group can then react rapidly with previously unreacted vinyl sulfone groups, either in free solution or in pendant form. The amino acid amine or side group (such as thiol of cysteine) can react rapidly with vinyl sulfone groups at alkaline conditions. This makes the waste stream less hazardous and quenches the pendant crosslinker groups. The gel can then be acid-neutralized and processed as desired or with standard manufacturing processes.

### BRIEF DESCRIPTION OF THE DRAWINGS

FIG. 1 (A) depicts a schematic for the preparation of vinyl crosslinked polymers. FIG (B) depicts schematics for the preparation of divinyl sulfone (DVS) crosslinked hyaluronic acid (HA) hydrogel. FIG (C), (D) and (E) depict schematics for the preparation of divinyl sulfone (DVS) crosslinked HA hydrogel showing crosslinking to various hydroxyls on HA.

FIGS. 2A and 2B depict chemical structures of exemplary DVS-crosslinked hyaluronic acid with a vinyl sulfone pendant group.

FIG. 3 depicts a chemical structure of an exemplary DVS-crosslinked hyaluronic acid with a lysine-quenched pendant group.

FIG. 4 depicts a chemical structure of an exemplary DVS-crosslinked hyaluronic acid with a cysteine-quenched pendant group.

FIG. 5 (A) depicts an exemplary crosslinked polymer with a quenched pendant group and a vinyl pendant group. FIG. 5 (B) depicts a chemical structure of an exemplary DVS-crosslinked polymer with a quenched pendant group and a vinyl pendant group.

FIG. 6 depicts a chemical structure of an exemplary 2-arm PEG vinyl sulfone-crosslinked polymer with a quenched pendant group and a vinyl pendant group.

FIG. 7 depicts a chemical structure of an exemplary 4-arm PEG vinyl sulfone-crosslinked polymer with a quenched pendant group and a vinyl pendant group.

FIG. 8 is a chart comparing concentrations of residual pendant vinyl sulfone groups on DVS-crosslinked HA gels following reaction with quenching agents: cysteine, glycine, lysine cysteamine and control (no quenching agent). The units are micromoles of vinyl sulfone per milliliter of gel. All four quenching agents resulted in a decrease in residual vinyl sulfone concentrations.

FIG. 9 is a chart comparing concentrations, analyzed through a modified Ellman's Assay, of residual pendant vinyl sulfone groups on DVS-crosslinked HA gels, with a quenching step and without a quenching step (control). Gels were quenched with cysteine at a molar ratio (cysteine to divinyl sulfone) of 1:100 for 5 minutes at 30° C. at a pH of approximately 12.7 (0.1 Molar Sodium Hydroxide in distilled water). (A) Residual vinyl sulfone concentration (including residual DVS groups and pendant VS groups) is substantially decreased with the additional quenching step. (B) Concentration of residual/pendant vinyl sulfone groups can be calculated on the basis of hyaluronic acid content in solution, showing the same effect of the quenching agent on reducing the residual vinyl sulfone concentration.

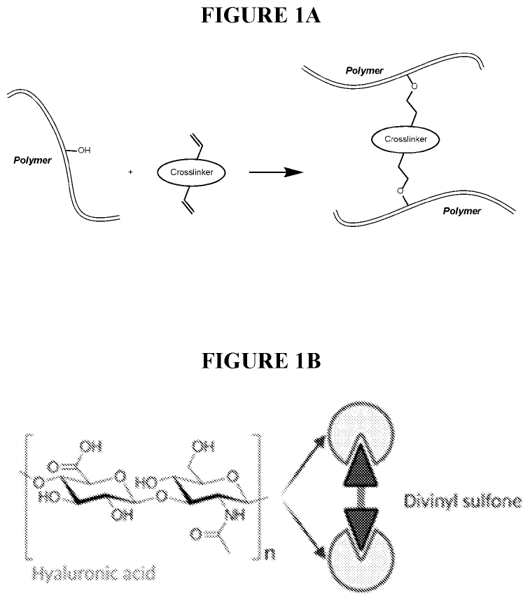

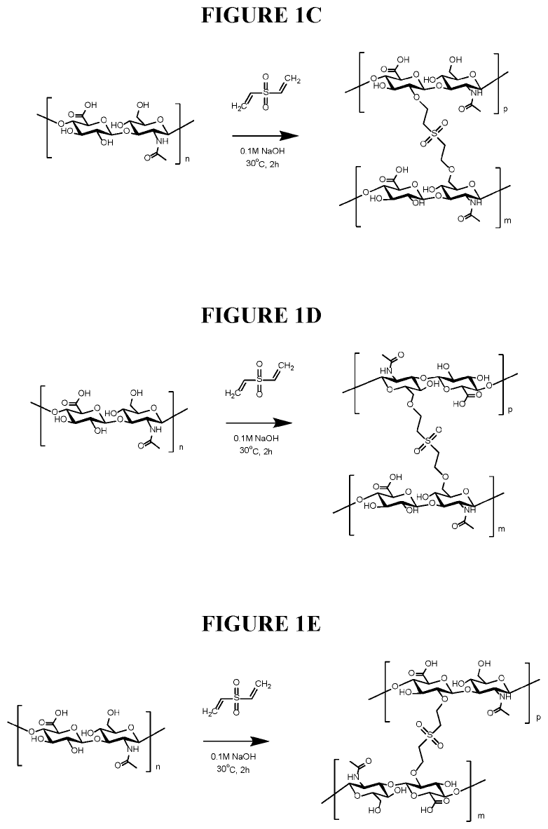

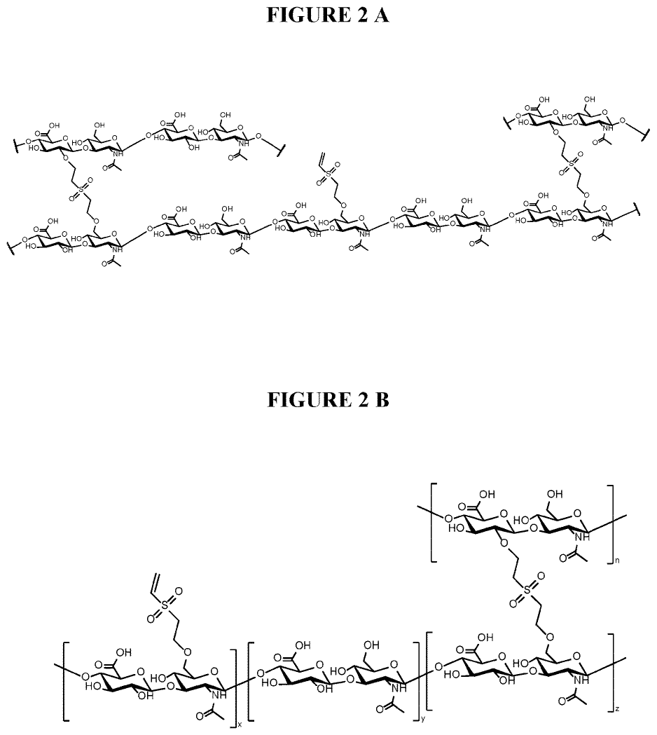

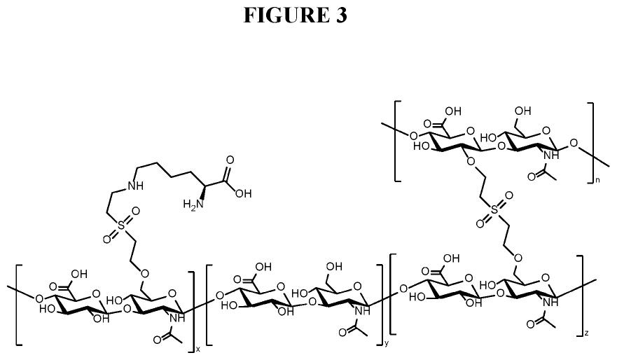

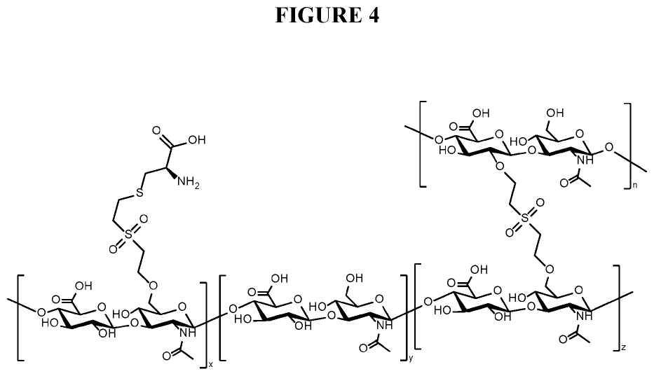

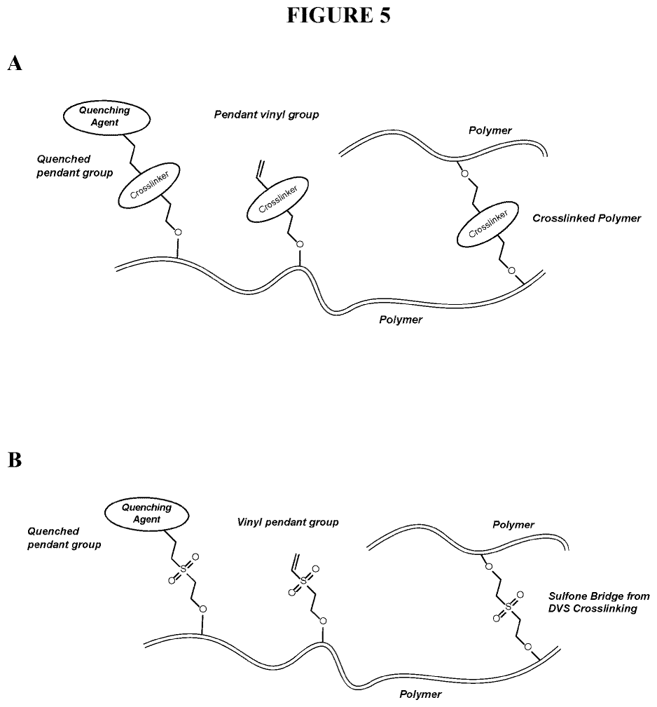

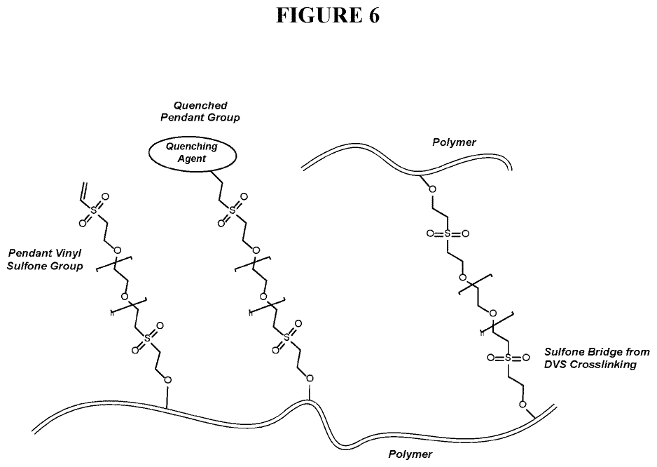

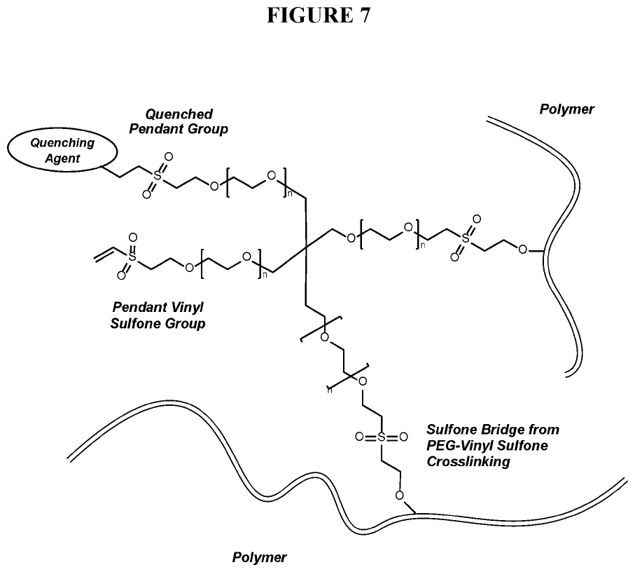

### DETAILED DESCRIPTION OF THE DISCLOSURE

Unless otherwise defined herein, scientific and technical terms used in connection with the present invention shall have the meanings that are commonly understood by those of ordinary skill in the art. In case of conflict, the present specification, including definitions, will control. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated.

### INCORPORATION BY REFERENCE

All publications, patents and other references mentioned herein are hereby incorporated by reference in their entireties.

### Definitions

The following terms, unless otherwise indicated, shall be understood to have the following meanings:

Throughout this specification and claims, the word “comprise” or variations such as “comprises” or “comprising”, will be understood to imply the inclusion of a stated integer or group of integers but not the exclusion of any other integer or group of integers.

The term “a” and “an” refers to one or to more than one (i.e., to at least one) of the grammatical object of the article. By way of example, “an element” means one element or more than one element.

As used herein, “about” or “approximately” can mean within an acceptable error range, plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein, the term “hydrogel” is a type of “gel,” and refers to a water-swellable polymeric matrix, consisting of a three-dimensional network of macromolecules (e.g., hydrophilic polymers, hydrophobic polymers, blends thereof) held together by covalent or non-covalent crosslinks that can absorb a substantial amount of water (e.g., 50%, 60% 70%, 80%, 90%, 95%, 96%, 97%, 98%, 99% or greater than 99% per unit of non-water molecule) to form an elastic gel. The polymeric matrix may be formed of any suitable synthetic or naturally occurring polymer material. As used herein, the term “gel” refers to a solid three-dimensional network that spans the volume of a liquid medium and ensnares it through surface tension effects.

The term “crosslinked” herein refers to a composition containing intramolecular and/or intermolecular crosslinks, whether arising through covalent or noncovalent bonding, and may be direct or include a cross-linker. “Noncovalent” bonding includes both hydrogen bonding and electrostatic (ionic) bonding.

As used herein, the term “concentration” in the context of divinyl sulfone (DVS) refers to a ratio of DVS molecules per hyaluronic acid (HA) repeat subunit. DVS concentration may also refer to the concentration of DVS calculated as the ratio of DVS molecules per hyaluronic acid (HA) disaccharide subunit hydroxyl group (with 4 hydroxyls per HA repeating subunit). DVS concentration may be represented as the multiple of DVS molecules to hydroxyl groups in a given sugar polymer (e.g., 1×, 2×, etc.).

As used herein, the term “Gelation Concentration,” refers to the concentration by weight of the reactant under the starting reaction conditions prior to and/or during gelation. Where more than one starting material is used in the reaction, Initial Concentration refers to the total concentration by weight of the starting materials, per volume, under the starting reaction conditions. (e.g., mg/mL).

As used herein, the term “pendant” group” refers to an unreacted functional group of a crosslinker. In various embodiments, a pendant group is formed when a crosslinking molecule has at least one, but not all, of its reactive sites that are reacted with the gel components, so that the crosslinking molecule is bound yet still retains at least one unreacted functional group. A pendent group comprising an unreacted functional group may also be referred to as a “reactive pendant group.” In various aspects, a pendant group may be formed by an incomplete crosslinking reaction.

In a non-limiting example, a pendant group may be formed when only one functional groups of difunctional crosslinker reacts with a molecular chain. Pendant groups can also occur with multi-functional crosslinking agents, such as a 4-arm PEG molecule with each arm containing a vinyl sulfone group. For such a molecule, if only 1 functional group is reacted, there are three remaining pendant groups; if 2 functional groups are reacted, there are two remaining pendant functional groups, if 3 functional groups are reacted, there is one remaining pendant functional group.

As used herein, the term “quenching agent” refers to a reagent that reacts with unreacted pendant functional groups. In a non-limiting example, a quenching agent refers to a reagent that reacts to reactive pendant groups.

### Crosslinking Agent

In various aspects, the crosslinking agent comprises a vinyl sulfone group. In various aspects, the crosslinking agent comprises multimers of a vinyl sulfone group. In various embodiments, the crosslinking agent comprises two or more vinyl sulfone groups. In various embodiments, the crosslinking agent comprises one, two, three or four vinyl sulfone groups. In various embodiments, the crosslinking agent comprises two, three or four vinyl sulfone groups. In various embodiments, the crosslinking agent comprises two vinyl sulfone groups. In various embodiments, the crosslinking agent comprises four vinyl sulfone groups. In various embodiments, the crosslinking agent comprises divinyl sulfone (DVS).

In various aspects, the crosslinking agent is a comprises two different functional groups. In various embodiments, wherein the crosslinking agent comprises two different a vinyl sulfone group, the crosslinking agent comprises at least one vinyl sulfone group.

In various embodiments, the crosslinking agent comprises a polyethylene glycol comprising a vinyl sulfone group (a “PEG-VS”). In various embodiments, the crosslinking agent comprises a polyethylene glycol comprising two vinyl sulfone groups (a “PEG-DVS”). In various embodiments, the crosslinking agent comprises a polyethylene glycol comprising three vinyl sulfone groups (a “PEG-tri-VS”). In various embodiments, the crosslinking agent comprises a polyethylene glycol comprising four vinyl sulfone groups (a “PEG-tetra-VS”).

Most crosslinking agents of commercial interest are bifunctional, with two functional groups per linker to each individually react with different molecular chains, resulting in a network hydrogel. However, if only one of the functional groups reacts with a molecular chain, the result is a “pendant” group linked into the network hydrogel that retains a potentially bioactive functional group. This is particularly relevant for crosslinking large molecules like high molecular weight polysaccharide such as hyaluronic acid (HA) with short crosslinking agents such as BDDE or DVS. Once one reactive group of a given crosslinker molecule reacts with an HA chain, the other reactive group of the molecule needs to come into close proximity to another HA chain in order to react. With short crosslinkers, two HA chains need to come into very close proximity to form functional crosslinks, which means overcoming steric hindrance and electrostatic repulsion between the HA chains resulting from a high negative charge of HA chains in the alkaline pH range used in typical crosslinking reactions.

Generally, a population of pendant crosslinking groups remain in the gel after crosslinking and purification, since purification processes such as dialysis cannot be used to remove chemicals that are covalently bonded to the network hydrogel.

BDDE-crosslinked gels have been dominant in the market over DVS crosslinked gels, with all currently marketed (in US) HA-based fillers utilizing BDDE crosslinking See, Faivre et al, Expert Review of Medical Devices, (2021), 18:12, 1175-1187. One major advantage of BDDE crosslinking is that such BDDE pendant groups can have greatly reduced toxicity after a steam sterilization process (autoclaving). In these gels, the pendant crosslinker epoxide groups can be hydrolyzed to a relatively inert form by the rapid hydrolysis kinetics during crosslinking and subsequent processing, such as autoclave sterilization (typically 121° C. or above). However, unlike with BDDE, the vinyl sulfone groups such as that of DVS do not hydrolytically degrade during autoclave sterilization because DVS lacks hydrolysable functional groups (Lyman W J et al; Handbook of Chemical Property Estimation Methods. Washington, DC: Amer Chem Soc pp. 7-4, 7-5 (1990)). Thus, a plurality of reactant pendant groups remain intact after the typical terminal sterilization autoclave cycle for DVS-crosslinked gels.

Several scientific review publications each point to concerns about the safety profile for DVS-crosslinked gels when used to treat human subjects.

“Some efforts have been made to use other crosslinkers. However, in the worst case these attempts have resulted in preclinical, clinical, or commercial failures. In the best case, they could not outperform BDDE and its safety and efficacy records.” (See, Faivre et al, (2021) Crosslinking hyaluronic acid soft-tissue fillers: current status and perspectives from an industrial point of view, Expert Review of Medical Devices, 18:12, 1175-1187).

“While various substances are available for use as cross-linking agents, including 1,4-butanediol diglycidyl ether (BDDE), divinyl sulfone (DVS), and biscarbodiimide (BCDI), BDDE is the cross-linking agent by far the most widely used in the commercial production of HA fillers owing to its unrivaled low toxicity profile. The median lethal dose (LD50) of BDDE administered orally to rats is only 1134 mg/kg, compared with 32 mg/kg for DVS.” (See, K. Seo, Facial Volumization with Fillers, Springer Singapore, 2021, Pages 38-39.)

“The relatively high biocompatibility of HAHMDA hydrogels might be ascribed to the nontoxic amine residues in contrast to the possible toxic remaining groups of BDDE in Restylane and DVS in HA-DVS hydrogels (Hylaform). The amine pendant groups within HA-HMDA hydrogels might be advantageous for cellular attachment and proliferation” See, J. Yeom et al. “Effect of cross-linking reagents for hyaluronic acid hydrogel dermal fillers on tissue augmentation and regeneration.” Bioconjugate chemistry 21.2 (2010): 240-247).

Risk of residual crosslinkers has been mitigated by others only by acid-quenching to halt the modification of HA (dropping pH from the extremely alkaline pH of 12 or above, to neutral or acidic pH). This halts the further reaction of vinyl sulfone with the deprotonated hydroxyls on the HA backbone and dilutes out the unreacted crosslinking agent. The gels are then typically precipitated or dialyzed to remove residual crosslinker agents through passive diffusion. However, previously, there has been no specific step taken to reduce the toxicity of any remaining soluble crosslinker or pendant crosslinking groups.

Vinyl sulfone groups have broad reactivity, which could introduce biocompatibility concerns if a high concentration of such reactive groups remain intact in the gel when implanted into or onto the body. Thus, safety concerns are a major impediment to creating innovative products using alternative crosslinkers to BDDE, especially residual crosslinker residuals and residual crosslinker pendant groups. The residual groups when using crosslinkers like DVS are both more hazardous than those from BDDE, but also more likely to remain in the device after manufacturing. Further mitigation has been needed.

### Reducing Toxicity

In various aspects, the present disclosure describes a method of reducing toxicity of a divinyl sulfone crosslinked material comprising pendant vinyl groups, said method comprising the step of: reacting the pendant vinyl sulfone group with a quenching agent whereby the pendant vinyl sulfone group is converted to a less toxic material. This present disclosure also reduces the toxicity of any unreacted, free-floating DVS that may remain in the gel even after processing. Without being bound by theory, the compositions of the present converted to a less toxic compositions because the materials are converted to groups that lacks or has limited reactivity to crosslink or alkylate proteins and biological constituents in vivo. In various embodiments, a “less toxic material” is a material that lacks or that has limited reactivity to crosslink or alkylate proteins and biological constituents in vivo. In various embodiments, “reducing toxicity” means limiting reactivity of a material to crosslink or alkylate proteins and biological constituents in vivo.

### Quenching Agent

In various embodiments, quenching agents comprise one or more nucleophiles, or Lewis-bases. Non-limiting examples of nucleophiles include thiols (also known as sulfhydryl groups), amines, hydroxyls, or halogens. In various embodiments, quenching agents comprise molecules that comprise thiols (also known as sulfhydryl groups), amines, hydroxyls, or halogens. In various embodiments, the halogen is fluorine, chlorine, bromine, or iodine.

In various embodiments, the quenching agents comprise one or more amine groups. In various embodiments, the quenching agents comprise one or more thiol groups. In various embodiments, the quenching agents comprise one or more hydroxyl groups. In various embodiments, the quenching agents comprise any combination of amine, thiol or hydroxyl groups.

In various embodiments, the quenching agent comprise two or more nucleophiles. The two or more nucleophiles may be the same or different. In various embodiments, quenching agent comprises an amine and a thiol.

In various embodiments, quenching agent comprises two or more amines.

In various embodiments, quenching agent comprises two or more thiols.

In various embodiments, quenching agent comprises two or more hydroxyls.

In various embodiments, quenching agent comprises an amine and a thiol.

In various embodiments, quenching agent comprises an amine and a hydroxyl.

In various embodiments, quenching agent comprises a thiol and a hydroxyl.

In various embodiments, the quenching agent comprises a polyamine molecule.

In various embodiments, the quenching agent comprises a polythiol molecule. In various embodiments, the quenching agent comprises a polyhydroxyl molecule.

In various embodiments, the quenching agent comprises more than one nucleophile.

In various embodiments, the quenching agent comprises two or more nucleophiles, wherein at least one nucleophile comprises an amine.

In various embodiments, the quenching agent comprises two or more nucleophiles, wherein at least one nucleophile comprises an amine, wherein the amine remains available for subsequent reactions following the quenching reaction.

In various embodiments, the amine in a primary amine following the quenching reaction.

In various embodiments, the quenching reaction forms a quenched pendant group, wherein the quenched pendant group comprises an amine, thiol or hydroxyl. In various embodiments, the quenched pendant group comprises an amine. In various embodiments, the quenched pendant group comprises a primary amine. In various embodiments, the quenched pendant group comprises a thiol. In various embodiments, the quenched pendant group comprises a primary thiol. In various embodiments, the quenched pendant group comprises an hydroxyl. In various embodiments, the quenched pendant group comprises a primary hydroxyl. Exemplary quenching agents include, but are not limited to, cysteamine, ethylenediamine (EDA) and amino acids,

In various embodiments, amino acids include, but are not limited to, cysteine (with reactive thiol group), lysine (with primary amines), arginine, histidine, glycine, glutamate or aspartate (anionic), leucine (increasing hydrophobicity), and asparagine (amide), methionine, serine (hydroxyl) and threonine (hydroxyl).

In various embodiments, amino acids include, but are not limited to, essential amino acids, non-essential amino acids, and amino acid derivatives.

A “non-essential” amino acid residue, as used herein, is an amino acid residue present in a wild-type sequence of a polypeptide that can be altered without abolishing or substantially altering essential biological or biochemical activity (e.g., receptor binding or activation) of the polypeptide.

An “essential” amino acid residue, as used herein, is an amino acid residue present in a wild-type sequence of a polypeptide that, when altered, results in abolishing or a substantial reduction in the polypeptide's essential biological or biochemical activity (e.g., receptor binding or activation).

In various embodiments, amino acids include any isomer of the amino acid. In various embodiments, amino acids or amino acid analogs are racemic. In various embodiments, the L-isomer of the amino acid is used. In various embodiments, the D-isomer of the amino acid is used. In various embodiments, the amino acid comprises chiral centers that are in the R or S configuration. In various embodiments, the chiral center is in the R configuration. In various embodiments, the chiral center is in the S configuration.

In various embodiments, amino acids include, but are not limited to, amino acids comprising unblocked nucleophilic side chains. In various embodiments, amino acids include, but are not limited to, amino acids comprising an amino group of a β-amino acid is substituted with a protecting group. Amino protecting groups include but not limited to, tert-butoxycarbonyl (BOC group), 9-fluorenylmethyloxycarbonyl (FMOC), and toluenesulfonyl (tosyl, Ts). In various embodiments, amino acids comprise an amino group substituted with a protecting group selected from the group consisting of tert-butoxycarbonyl (BOC group), 9-fluorenylmethyloxycarbonyl (FMOC), and toluenesulfonyl (tosyl, Ts). In various embodiments, the carboxylic acid functional group of a β-amino acid is protected. In various embodiments, the carboxylic acid is protected as an ester derivative. In various embodiments, the nucleophilic side chain is protected. In various embodiments a salt of the amino acid analog is used. In various embodiments the salt is a pharmaceutically acceptable salt.

In various embodiments, the amino acid comprises a capping group. The term “capping group” refers to the chemical moiety occurring at either the carboxy or amino terminus of a polypeptide chain. A capping group of a carboxy terminus includes an unmodified carboxylic acid (i.e., —COOH) or a carboxylic acid with a substituent. For example, the carboxy terminus can be substituted with an amino group to yield a carboxamide at the C-terminus. Various substituents include but are not limited to primary and secondary amines, including but not limited to, pegylated secondary amines. A capping group of an amino terminus includes an unmodified amine (i.e., —NH₂) or an amine with a substituent. For example, the amino terminus can be substituted with an acyl group to yield a carboxamide at the N-terminus. Various substituents include but are not limited to substituted acyl groups, including C₁-C₆ carbonyls, C₇-C₃₀ carbonyls, and pegylated carbamates. Polypeptides can also be used as quenching agents.

In various embodiments, the quenching agent comprises a polypeptide. In various embodiments, the polypeptide comprises a bioactive sequences.

In various embodiments, the bioactive sequence comprises cell recognition or cell attachment activity. In various embodiments, the bioactive sequence comprises a sequence present in a cell-attachment domain of a biological polymer. In various embodiments, the bioactive sequence comprises a sequence present in a cell-attachment domain of a biological polymer comprising fibronectin, vitronectin, laminin, or collagen. In various embodiments, the bioactive sequence comprises Arg-Gly-Asp (RGD). In various embodiments, the bioactive sequence comprises Arg-Gly-Asp-Ser (RGDS), Arg-Gly-Asp-Val (RGDV), Arg-Gly-Asp-Thr (RGDT), and Ile-Lys-Val-Ala-Val (IKVAV).

In various embodiments, the quenching agent comprises an amine (also referred to as an amine quenching agent). In various embodiments, a quenching agent includes, but in not limited to, an amine quenching agent.

In various embodiments, the quenching agent comprises a polyamine. In various embodiments, polyamines include, but are not limited to, ethylenediamine (EDA), polyethylene amines, putrescine, spermine, thermospermine, and spermidine.

In various embodiments, the quenching agent comprises a thiol. In various embodiments, thiols include, but are not limited to, propane-1,3-dithiol and dimercaprol.

In various embodiments, the quenching agent comprises an aminothiol. An aminothiol, as referred to herein, is a molecule containing both an amine and a thiol functional groups. In various embodiments, include, but are not limited to, cysteamine, and ((aminopropyl)amino) ethanethiol, 3-(methylamino)-2-((methylamino)methyl) propane-1-thiol or 3-amino-2-(aminomethyl) propane-1-thiol.

An advantage of using amino acids, amines, polyamines or their derivatives, as quenching agents is the resulting ability to encourage cellular attachment and migration. Without being bound by theory, incorporation of amino acids such as lysine or amine-containing molecules such as EDA may result in amines being present on the polymer backbone, thereby encouraging cellular attachment and migration. Cellular attachment and migration may be desirable for cell permeable gels.

### Quenching Reaction pH

Without being bound by theory, functional groups with higher reactivity allow for faster reaction kinetics. For example, lower reaction times is one example of lower reaction kinetics factor that may allow for a broader range of pH conditions to be used. In various embodiments, quenching agents comprise highly reactive functional groups. Without being bound by theory, a quenching agent is more reactive if it comprises a functional group that deprotonates more readily, or at a lower pH.

In various embodiments, the quenching agent is any suitable agent able to react as a nucleophile with a vinyl group during a Michael Addition reaction. In various embodiments, the quenching agent is any suitable agent able to react as a nucleophile with a vinyl sulfone group during a Michael Addition reaction. In various embodiments, the quenching agent comprises a functional group that deprotonates. In various embodiments, the quenching agent comprises a functional group that deprotonates readily. In various embodiments, the quenching agent comprises a functional group that deprotonates at a pH of less than 14. In various embodiments, the quenching agent comprises a functional group that deprotonates at a pH of less than 13. In various embodiments, the quenching agent comprises a functional group that deprotonates at a pH of less than 12. In various embodiments, the quenching agent comprises a functional group that deprotonates at a pH of less than 11. In various embodiments, the quenching agent comprises a functional group that deprotonates at a pH of less than 10. In various embodiments, the quenching agent comprises a functional group that deprotonates at a pH of less than 9. In various embodiments, the quenching agent comprises a functional group that deprotonates at a pH of less than 8.

An advantage of using a quenching agent comprising a more reactive or nucleophilic functional group species (compared to the groups being crosslinked, such as the hydroxyls of hyaluronic acid) such as a thiol or a primary amine, the quenching can alternatively be performed at lower pH, halting the further modification of HA with DVS and limiting the degradation of constituents due to their hydrolysis at high pH. Instead, the pH can be dropped to 10 or below before addition of quenching agent, as well as being reacted for a significantly longer time if needed for reducing the pendant concentration further. Thiols can react with the vinyl sulfone groups at neutral pH, so if using a quenching agent like cysteine, the pH can be dropped to near 7 and reacted for a long time to complete the pendant group quenching. (See: Yu Y. et al., Biomacromolecules 13.3 (2012): 937-942.)

In various embodiments, quenching agents comprise amines and/or thiols. Without being bound to theory, amines and thiols maximize reaction rates, thereby minimizing quenching time at high pH. A quenching pH that is above the pKa value for the quenching group (such as the ionized amine side chain and/or peptide amine group of an amino acid), but below the pH where substantial HA hydrolysis occurs, can allow for longer quenching times for more complete quenching while minimizing degradation of the HA backbone. For example, since the pKa of lysine's side chain amino group in water is 10.4 (Isom, Daniel G., et al. “Large shifts in pKa values of lysine residues buried inside a protein.” Proceedings of the National Academy of Sciences 108.13 (2011): 5260-5265), the amine group may be deprotonated and suitable for reaction with vinyl sulfone at a pH above 10.4 yet below the pH were substantial HA degradation occurs (pH>11). A high pH, such as 11 or above, and especially 13, is associated with HA degradation through hydrolytic cleavage of the HA backbone (A. Maleki, et al., “Effect of pH on the behavior of hyaluronic acid in dilute and semidilute aqueous solutions,” Macromolecular symposia. Vol. 274. No. 1. Weinheim: WILEY-VCH Verlag, 2008). Similarly, the pKa of cysteine's thiol side chain in water is 8.6, providing a suitable range of quenching pH below the onset pH of accelerated HA hydrolysis (G. Roos, et al., “Understanding the pKa of redox cysteines: The key role of hydrogen bonding.” Antioxidants & redox signaling 18.1 (2013): 94-127).

In various embodiments, the quenching reaction step is performed at a pH relative to the pKa of the quenching agent. In various embodiments, the quenching reaction step is performed at a pH equal to or above the pKa of the quenching agent. In various embodiments, the quenching reaction step is performed at a pH about equal to the pKa of the quenching agent. In various embodiments, the quenching reaction step is performed at a pH above the pKa of the quenching agent. In various embodiments, the quenching reaction step is performed at about a 1.0 pH unit (relative to pKa) above the pKa of the quenching agent. In various embodiments, the quenching reaction step is performed at about 2.0 pH units above the pKa of the quenching agent. In various embodiments, the quenching reaction step is performed at about 3.0 pH units above the pKa of the quenching agent. In various embodiments, the quenching reaction step is performed at a pH ranging from about 0 to about 1.0 pH units above the pKa of the quenching agent. In various embodiments, the quenching reaction step is performed at a pH ranging from about 0.1 to about 1.0 pH units above the pKa of the quenching agent. In various embodiments, the quenching reaction step is performed at a pH ranging from about 0.5 to about 1.0 pH units above the pKa of the quenching agent.

In one non-limiting example, lysine has a side-chain amine with a pKa of about 10.8. In various embodiments, a quenching step using lysine as a quenching agent is performed at a pH ranging from about 12 to about 13. In another non-limiting example, cysteine has a side-chain thiol with a pKa of about 8.3. In various embodiments, a quenching step using cysteine as a quenching agent is performed at a pH ranging from about 9 to about 10.

In various embodiments, the quenching reaction step is performed at a pH lower than about 12. In various embodiments, the quenching reaction step is performed at a pH lower than about 11. In various embodiments, the quenching reaction step is performed at a pH lower than about 10.0. In various embodiments, the quenching reaction step is performed at a pH lower than about 9.0. In various embodiments, the quenching reaction step is performed at a pH lower than about 8.0.

In various embodiments the quenching reaction is performed at a pH ranging from about 8.0 to about 12.0. In various embodiments the quenching reaction is performed at a pH ranging from about 9.0 to about 11.0. In various embodiments the quenching reaction is performed at a pH ranging from about 8.0 to about 9.0. In various embodiments the quenching reaction is performed at a pH ranging from about 9.0 to about 10.0. In various embodiments the quenching reaction is performed at a pH ranging from about 10.0 to about 11.0.

In one embodiment the quenching reaction is performed at a pH ranging from about 8.6 to about 12.0. In one embodiment the quenching reaction is performed at a pH ranging from about 10.4 to about 11.0. In one embodiment the quenching reaction is performed at a pH ranging from about 10.4 to about 11.4. In one embodiment the quenching reaction is performed at a pH ranging from about 8.6 to about 10.

In one embodiment the quenching reaction is performed at a pH of less than about 10. In one embodiment the quenching reaction is performed at a pH of about 13. In one embodiment the quenching reaction is performed at a pH of about 12. In one embodiment the quenching reaction is performed at a pH of about 11. In one embodiment the quenching reaction is performed at a pH of about 10. In one embodiment the quenching reaction is performed at a pH of about 9.

In one embodiment the quenching reaction is performed at a pH of about 10. In one embodiment the quenching reaction is performed at a pH of about 13. In one embodiment the quenching reaction is performed at a pH of about 12. In one embodiment the quenching reaction is performed at a pH of about 11. In one embodiment the quenching reaction is performed at a pH of about 10. In one embodiment the quenching reaction is performed at a pH of about 9. In one embodiment the quenching reaction is performed at a pH of about 10. In one embodiment the quenching reaction is performed at a pH of about 8.

In various embodiments, the quenching agent is present in the quenching step at a concentration ranging from about 0.01 to about 100 times the concentration of DVS initially used for the crosslinking reaction. In various embodiments, the quenching agent is present in the quenching step at a concentration ranging from about 0.01 to about 1 times the concentration of DVS used for the crosslinking reaction.

In various embodiments, the quenching reaction comprises a quenching agent and a DVS at a molar ratio ranging from about 100:1 to about 1:100 (quenching agent: DVS concentration used in the crosslinking reaction). In various embodiments, the quenching reaction comprises a quenching agent and a DVS at a molar ratio ranging from about 100:1 to about 1:1 (quenching agent: DVS concentration used in the crosslinking reaction). In various embodiments, the quenching reaction comprises a quenching agent and a DVS at a molar ratio ranging from about 1:1 to about 1:100 (quenching agent: DVS concentration used in the crosslinking reaction). In various embodiments, the quenching reaction comprises a quenching agent and a DVS at a molar ratio ranging from about 1:1 to about 1:90 (quenching agent: DVS concentration used in the crosslinking reaction). In various embodiments, the quenching reaction comprises a quenching agent and a DVS at a molar ratio ranging from about 1:1 to about 1:80 (quenching agent: DVS concentration used in the crosslinking reaction).

In various embodiments, the quenching reaction comprises a quenching agent and a DVS at a molar ratio ranging from about 1:1 to about 1:70 (quenching agent: DVS). In various embodiments, the quenching reaction comprises a quenching agent and a DVS at a molar ratio ranging from about 1:1 to about 1:60 (quenching agent: DVS). In various embodiments, the quenching reaction comprises a quenching agent and a DVS at a molar ratio ranging from about 1:1 to about 1:50 (quenching agent: DVS). In various embodiments, the quenching reaction comprises a quenching agent and a DVS at a molar ratio ranging from about 1:1 to about 1:40 (quenching agent: DVS). In various embodiments, the quenching reaction comprises a quenching agent and a DVS at a molar ratio ranging from about 1:1 to about 1:30 (quenching agent: DVS). In various embodiments, the quenching reaction comprises a quenching agent and a DVS at a molar ratio ranging from about 1:1 to about 1:20 (quenching agent: DVS). In various embodiments, the quenching reaction comprises a quenching agent and a DVS at a molar ratio ranging from about 1:1 to about 1:10 (quenching agent: DVS). In various embodiments, the quenching reaction comprises a quenching agent and a DVS at a molar ratio ranging from about 1:1 to about 1:5 (quenching agent: DVS). In various embodiments, the quenching reaction comprises a quenching agent and a DVS at a molar ratio ranging from about 1:1 to about 1:2 (quenching agent: DVS).

In various embodiments, the quenching reaction comprises a quenching agent and a DVS at a molar ratio of about 1:1, (quenching agent: DVS). In various embodiments, the quenching reaction comprises a quenching agent and a DVS at a molar ratio of about 1:10, (quenching agent: DVS). In various embodiments, the quenching reaction comprises a quenching agent and a DVS at a molar ratio of about 1:100, (quenching agent: DVS).

### Concentration of Unquenched Pendant Groups

In various aspects, the crosslinked polymer comprises a concentration of unquenched pendant groups of at most about 10 micromoles/mL. In various aspects, the crosslinked polymer comprises a concentration of unquenched pendant groups of at most about 5 micromoles/mL. In various aspects, the crosslinked polymer comprises a concentration of unquenched pendant groups of at most about 2.5 micromoles/mL. In various aspects, the crosslinked polymer comprises a concentration of unquenched pendant groups of at most about 2 micromoles/mL. In various aspects, the crosslinked polymer comprises a concentration of unquenched pendant groups of at most about 1.5 micromoles/mL. In various aspects, the crosslinked polymer comprises a concentration of unquenched pendant groups of at most about 1.2 micromoles/mL. In various aspects, the crosslinked polymer comprises a concentration of unquenched pendant groups of at most about 1.0 micromoles/mL.

In various aspects, the crosslinked polymer comprises a concentration of unquenched pendant groups ranging from about 0.1 micromoles/mL to about 10 micromoles/mL. In various aspects, the crosslinked polymer comprises a concentration of unquenched pendant groups ranging from about 0.1 micromoles/mL to about 1 micromole/mL. In various aspects, the crosslinked polymer comprises a concentration of unquenched pendant groups ranging from about 1 micromole/mL to about 10 micromoles/mL. In various aspects, the crosslinked polymer comprises a concentration of unquenched pendant groups ranging from about 1 micromole/mL to about 5 micromoles/mL. In various aspects, the crosslinked polymer comprises a concentration of unquenched pendant groups ranging from about 1 micromole/mL to about 2 micromoles/mL.

### Quenching Reaction Conditions

In various embodiments, a quenching agent (a nucleophile such as lysine, glycine, cysteine, or equivalent) is added to the reactant mixture at the end of gel crosslinking reaction, but before the acid neutralization. In various embodiments, the quenching agent is allowed time to diffuse and react with residual vinyl sulfone groups, prior to acid-neutralizing the reaction. In various embodiments, the quenching reaction time ranges from at least about 1 minute to about 1 hour.

In various embodiments, the quenching reaction time ranges from at least about 1 minute to about 1 hour at room temperature or above. In various embodiments, the quenching reaction time ranges from at least about 5 minute to about 15 minutes at room temperature or above.

In various embodiments, the quenching reaction takes place at a temperature below about the boiling point of an aqueous solution or mixture. In various embodiments, the quenching reaction takes place at a temperature below about the boiling point of water. In various embodiments, the quenching reaction takes place at a temperature below about 100° C.

In various embodiments, the quenching reaction takes place at a temperature ranging from about 25° C. to about 90° C. . . . In various embodiments, the quenching reaction takes place at a temperature ranging from about 25° C. to about 80° C. . . . In various embodiments, the quenching reaction takes place at a temperature ranging from about 25° C. to about 70° C. . . . In various embodiments, the quenching reaction takes place at a temperature ranging from about 25° C. to about 60° C. . . . In various embodiments, the quenching reaction takes place at a temperature ranging from about 25° C. to about 50° C. In various embodiments, the quenching reaction takes place at a temperature ranging from about 25° C. to about 40° C. In various embodiments, the quenching reaction takes place at a temperature ranging from about 25° C. to about 30° C.

In various embodiments, the quenching reaction takes place at about room temperature or above. In various embodiments, the quenching reaction takes place at about room temperature.

In various embodiments, the quenching reaction takes place at room temperature or above, especially 5-15 minutes, though up to several hours when at or under 25° C.

In various embodiments, the quenching reaction takes place at about 0° C. In various embodiments, the quenching reaction takes place below about 0° C.

### Reduction in Residual Pendant Vinyl Sulfone Groups

In various embodiments, the composition comprises a diminished number of pendant vinyl sulfone groups.

In various aspects, quenching of pendant vinyl sulfone groups results in a reduction of the number of residual unreacted pendant vinyl sulfone groups.

In various embodiments, the crosslinked polymer comprises residual sulfone groups at a concentration of at most about 0.1 μg/mL. In various embodiments, the crosslinked polymer comprises residual sulfone groups at a concentration of at most about 0.5 μg/mL, 1.0 μg/mL, 1.5 μg/mL, 2.0 μg/mL.

In various embodiments, the crosslinked polymer comprises residual sulfone groups at a concentration of at most about 1.7 μg/mL.

In various embodiments, the crosslinked polymer comprises residual sulfone groups at a concentration of at most about 1.4 μmol/mL In various embodiments, the crosslinked polymer comprises residual sulfone groups at a concentration of at most about 1.0 μg/mL. In various embodiments, the crosslinked polymer comprises residual sulfone groups at a concentration of at most about 0.5 μg/mL.

In various embodiments, the crosslinked polymer comprises residual sulfone groups at a concentration ranging from about 0.1 μmol/mL to about 2.0 μg/mL. In various embodiments, the crosslinked polymer comprises residual sulfone groups at a concentration ranging from about 0.1 μmol/mL to about 1.5 g/mL. In various embodiments, the crosslinked polymer comprises residual sulfone groups at a concentration ranging from about 0.1 μmol/mL to about 1.0 μg/mL. In various embodiments, the crosslinked polymer comprises residual sulfone groups at a concentration ranging from about 0.5 μmol/mL to about 1.0 μg/mL. In various embodiments, the crosslinked polymer comprises residual sulfone groups at a concentration ranging from about 0.5 μmol/mL to about 1.5 μg/mL. In various embodiments, the crosslinked polymer comprises residual sulfone groups at a concentration ranging from about 0.5 μmol/mL to about 1.4 μg/mL.

In various embodiments, the methods of the present disclosure result in a reduction of residual vinyl sulfone groups by a range of about 10% to about 99.9%.

In various embodiments, the methods of the present disclosure result in a reduction of residual vinyl sulfone groups by a range of about 10% to about 80%, a range of about 10% to about 70%, a range of about 10% to about 60%, a range of about 10% to about 50%, a range of about 10% to about 40%, a range of about 10% to about 30%, or a range of about 10% to about 20%.

In various embodiments, the methods of the present disclosure result in a reduction of residual vinyl sulfone groups by at least about 10%.

In various embodiments, the methods of the present disclosure result in a reduction of residual vinyl sulfone groups by at least about 10%, 20%, 30%, 40%, 50%, 60%, 70%, 80%, or 90%.

In various embodiments, the methods of the present disclosure result in a reduction of residual vinyl sulfone groups by at least about 20%. In various embodiments, the methods of the present disclosure result in a reduction of residual vinyl sulfone groups by at least about 18%. In various embodiments, the methods of the present disclosure result in a reduction of residual vinyl sulfone groups by at least about 50%. In various embodiments, the methods of the present disclosure result in a reduction of residual vinyl sulfone groups by at least about 60%. In various embodiments, the methods of the present disclosure result in a reduction of residual vinyl sulfone groups by at least about 67%.

### Concentrations

### Concentration of DVS-Crosslinked Polymer

In various embodiments, the reaction conditions for crosslinking the polymer comprises divinyl sulfone present in an amount ranging from about 1:1 to about 5:1 divinyl sulfone molecules per monomer subunit, from about 2:1 to about 5:1 divinyl sulfone molecules per monomer subunit, from about 3:1 to about 5:1 divinyl sulfone molecules per monomer subunit, from about 3.5:1 to about 4:1 divinyl sulfone molecules per hyaluronic acid subunit.

In various embodiments, the reaction conditions for the crosslinked polymer comprises divinyl sulfone present in an amount of about 3.5:1 divinyl sulfone molecules per monomer subunit, about 4:1 divinyl sulfone molecules per monomer subunit, or about 4.5:1 divinyl sulfone molecules per hyaluronic acid subunit.

In various embodiments, the reaction conditions for the crosslinked polymer comprises divinyl sulfone present in an amount ranging from about 1:1 to about 5:1 divinyl sulfone molecules per hyaluronic acid subunit, from about 2:1 to about 5:1 divinyl sulfone molecules per hyaluronic acid subunit, from about 3:1 to about 5:1 divinyl sulfone molecules per hyaluronic acid subunit, from about 3.5:1 to about 4:1 divinyl sulfone molecules per hyaluronic acid subunit.

In various embodiments, the reaction conditions for the crosslinked polymer comprises divinyl sulfone present in an amount of about 3.5:1 divinyl sulfone molecules per hyaluronic acid subunit, about 4:1 divinyl sulfone molecules per hyaluronic acid subunit, or about 4.5:1 divinyl sulfone molecules per hyaluronic acid subunit.

### Concentration of DVS-Hydrogel

In various embodiments, the gelling conditions for the hydrogel comprises divinyl sulfone present in an amount ranging from about 1:1 to about 5:1 divinyl sulfone molecules per monomer subunit, from about 2:1 to about 5:1 divinyl sulfone molecules per monomer subunit, from about 3:1 to about 5:1 divinyl sulfone molecules per monomer subunit, from about 3.5:1 to about 4:1 divinyl sulfone molecules per hyaluronic acid subunit.

In various embodiments, the gelling conditions for the hydrogel comprises divinyl sulfone present in an amount of about 3.5:1 divinyl sulfone molecules per monomer subunit, about 4:1 divinyl sulfone molecules per monomer subunit, or about 4.5:1 divinyl sulfone molecules per hyaluronic acid subunit.

In various embodiments, the gelling conditions for the hydrogel comprises divinyl sulfone present in an amount ranging from about 1:1 to about 5:1 divinyl sulfone molecules per hyaluronic acid subunit, from about 2:1 to about 5:1 divinyl sulfone molecules per hyaluronic acid subunit, from about 3:1 to about 5:1 divinyl sulfone molecules per hyaluronic acid subunit, from about 3.5:1 to about 4:1 divinyl sulfone molecules per hyaluronic acid subunit.

In various embodiments, the gelling conditions for the hydrogel comprises divinyl sulfone present in an amount of about 3.5:1 divinyl sulfone molecules per hyaluronic acid subunit, about 4:1 divinyl sulfone molecules per hyaluronic acid subunit, or about 4.5:1 divinyl sulfone molecules per hyaluronic acid subunit.

### Polyethylene Glycol (PEG)

In various aspects, hydrogel materials may additionally comprise Polyethylene Glycol PEG (with hydroxyl ends). In various aspects, hydrogel materials may additionally comprise PEG to be incorporated as additional potential crosslinkers into the resulting gel.

In various embodiments, hyaluronic acid (HA) materials may additionally include PEG (with hydroxyl ends), to be incorporated into a hydrogel. incorporating PEG can be performed in the same reaction of the HA and DVS together. For example, PEG, DVS and HA can be reacted in situ.

Without being bound by theory, PEG can be used to control porosity of gels by increasing spacing between HA chains. This porosity can be tuned by using longer or shorter chains of PEG, and by controlling the PEG concentration, without being bound by theory, by doing this, the resulting crosslinked sugar polymer strands are held at greater or lesser distances apart (largely, by controlling the PEG chain lengths), and/or crosslinked together at greater or lesser frequencies (largely by controlling the PEG concentrations).

The PEG hydroxyls of each PEG can each react with a DVS molecule, which will result in a PEG divinyl sulfone structure, since there will be a vinyl sulfone on each leg.

Alternatively, in various embodiments, PEG can be capped with vinyl sulfone endgroups before reaction with a polymer. A PEG capped with a vinyl sulfone may be referred to as a “PEG-VS.” A PEG capped with two vinyl sulfone groups may be referred to as a “PEG-DVS”

Alternatively, the PEG may comprise other vinyl sulfone-nucleophilic reacting groups including, but not limited to, amines or thiols. (e.g., PEG-amine-vinyl sulfone or PEG-thio-vinyl sulfone).

Additionally, 4-arm (or branched), 8-arm, or alternate valences of PEG-OH can be used. In various embodiments, the PEG is a 2-arm PEG. In various embodiments, the PEG is a 4-arm PEG. In various embodiments, the PEG is a 2-arm PEG. In various embodiments, the PEG is an 8-arm PEG.

The arms of the PEG molecule can vary from hundreds of Daltons to hundreds of thousands of Daltons. In various embodiments, the PEG comprises a molecular weight ranging from about 1 kDa to about 1000 kDa. In various embodiments, the PEG comprises a molecular weight ranging from about 1 kDa to about 500 kDa. In various embodiments, the PEG comprises a molecular weight ranging from about 1 kDa to about 100 kDa. In various embodiments, the PEG comprises a molecular weight ranging from about 1 kDa to about 10 kDa.

The concentration of PEG component can be varied from about 1 mg/mL to about 100 mg/mL. In various embodiments, the concentration of PEG ranges from about 1 mg/mL to about 100 mg/mL. In various embodiments, the concentration of PEG ranges from about 10 mg/mL to about 100 mg/mL. In various embodiments, the concentration of PEG ranges from about 10 mg/mL to about 100 mg/mL. In various embodiments, the concentration of PEG ranges from about 1 mg/mL to about 10 mg/mL. In various embodiments, the concentration of PEG ranges from about 1 mg/mL to about 50 mg/mL. In various embodiments, the concentration of PEG ranges from about 50 mg/mL to about 100 mg/mL.

Additional Benefits of compositions and methods:

Besides quenching vinyl sulfone groups to improve the sugar polymer hydrogel's safety profile, the chemical quenching also provides an opportunity to further tune the gel properties through choice of quenching agent. This allows for conjugation of chemical groups to the hydrogel network after primary gel formation.

### Tuning Gel Properties

Quenching agents can be used to tune the overall gel properties, such as hydrophobicity/hydrophilicity, electronegativity, or other functions. For example, cysteine can increase the mucoadhesive properties of the gel, with cysteine-quenched gels being substantially tackier, qualitatively, when handling after production. For example, this tacky trait could be used to mitigate implant migration in vivo or for mucosal targeting of drug delivery. Additional sulfur groups could also result in a more immunostimulatory gel, which could be useful for immunomodulation products. Methionine could be used as a quenching agent in a similar fashion. These agents, however, may not be preferentially chosen when adhesion is undesired, such as some surgical mesh use cases in which adhesion formations between two normally separate tissues may be undesirable.

### Electronegativity

With this invention, one can tune the electronegativity of the crosslinked sugar polymer backbone. For example, on my choose choice of groups with different electronegativity in their side chains, such as positively charged (such as histidine, lysine, arginine, polylysine, etc.), negatively charged (such as aspartate or glutamate), or uncharged (such as glycine, alanine, serine, threonine, asparagine, glutamine, etc.) to be used as crosslinking agents.

### Hydrophilicity/Hydrophobicity

With this invention, one can tune the hydrophilicity/hydrophobicity of the gel backbone to a degree with a choice of the hydrophilicity/hydrophobicity of the quenching agent. For example, one may quench the residual vinyl sulfone pendants with polar or ionic agents such as lysine or glutamate to increase a hydrogel's hydrophilicity, or quench with non-polar agents such as alanine or leucine (or other small molecules such as an aliphatic chain with an amine or thiol group) in order to increase a hydrogel's hydrophobicity.

### Adding Functionality

The quenching step can be used to provide additional functionality to the gel. The quenching step can provide additional functionality to the gel, by appending a functional moiety or functional agent to the gel.

In various embodiments, the functional agent comprises a biologically active agent. In various embodiments, the biologically active agent comprises a biologically active peptide. In various embodiments, the biologically active peptide is biologically active sequence. In various embodiments, the bioactive sequence comprises cell recognition or cell attachment activity. In various embodiments, the bioactive sequence comprises a sequence present in a cell attachment domain of a biological polymer. In various embodiments, the bioactive sequence comprises a sequence present in a cell attachment domain of a biological polymer comprising fibronectin, vitronectin, laminin, or collagen. In various embodiments, the bioactive sequence comprises Arg-Gly-Asp (RGD). In various embodiments, the bioactive sequence comprises Arg-Gly-Asp-Ser (RGDS), Arg-Gly-Asp-Val (RGDV), Arg-Gly-Asp-Thr (RGDT), and Ile-Lys-Val-Ala-Val (IKVAV).

In a non-limiting example, the quenching step can provide additional functionality to the gel, such as by appending a biologically active peptide sequence such as RGD or IKVAV,

In various embodiments, the quenching step can provide additional functionality by adding a functional group that allows for a further step of subsequent modification. For example, the quenching agent can comprise at least 1 group that can be used to react with vinyl sulfone, and at least 1 group that can be used thereafter to conjugate a second agent.

This two-step conjugation may be necessary if the intended cargo is incompatible with the potentially harsh alkaline conditions present at the quenching step. The target cargo can then be added after the reaction mixture's pH has been lowered to a compatible level. Alternatively, the target cargo can then be added after sterilization, or even at the point-of-care if the cargo is unsuitable for autoclave sterilization and/or long-term storage conditions.

### Toxicity of Waste Streams

Chemical quenching has the benefit of rendering unreactive one or more of the reactive groups of residual unbound crosslinking agents, uncrosslinked groups, which decreases the toxicity of the manufacturing waste streams.

### Durability of Hydrogels

The current trend of on-market sugar polymer hydrogels such as HA dermal fillers has been toward longer-lasting effects by making the gels more resistant to degradation after their implantation. This typically is accomplished by making denser gels with high HA concentrations and high crosslinking degrees. Furthermore, DVS crosslinking has been mostly superseded by BDDE crosslinking in the current dermal filler market.

Employing the use of amino acids as nucleophilic quenching agents can be used to obtain benefits in addition to the reduction of possible pendant VS crosslinker toxicities. Amino acid conjugation was thought to lead to cell-mediated hydrogel degradation and a shorter gel duration and thus a shorter clinical effect as the hydrogel backbone could increase and encourage cellular motility and material interaction. Cellular interaction is a major cause of hyaluronic acid degradation (particularly enzymatic cleavage of hyaluronic acid, such as through HYAL-2).

Also, it is unintuitive that such a small modification degree (only pendant vinyl sulfone groups are available for quencher modification) had such a large effect on resulting macroscopic gel properties such as gel tackiness.

In previous examples acid has been used to quench the DVS-crosslinking reaction. However, acid quench merely halts reactions between the vinyl sulfone groups and the hydrogel backbone. It does not have any direct effect upon the pendant vinyl sulfone groups.

For example, U.S. Pat. No. 8,481,080 describes making crosslinked HA gels with divinyl sulfone, with the DVS-crosslinking reaction followed by acidic treatment during the swelling process, to give final gel concentrations of 1.1%-1.4%, with no mention of any chemical quenching, residual vinyl sulfone concentrations, or pendant vinyl sulfone groups. In another example, US20050142152A1 uses acidic saline and PBS washes to remove impurities after DVS reaction, which is a solely acid quenching. (See: Y. Yu, et al., Biomacromolecules 13.3 (2012): 937-942.)

### Polymers

In various aspects, any polymer comprising one or more nucleophilic groups may be used. In various aspects, any polymer capable of reacting with vinyl groups may be used. In various aspects, any polymer capable of reacting with vinyl sulfone groups may be used. In various aspects, any polymer capable of reacting with divinyl sulfone groups may be used.

In various aspects, any polymer comprising one or more nucleophilic moieties that are capable of reacting with vinyl sulfone groups may be used.

In various embodiments, the polymer is a hydroxylated polymer. In various embodiments, the hydroxylated polymer comprises one or more hydroxyl groups. In various embodiments, the hydroxylated polymer comprises one or more hydroxyl groups per monomer unit.

In various embodiments, the hydroxylated polymer comprises 1, 2, 3, 4, 5, or 6 hydroxyl groups per monomer unit. In various embodiments, the hydroxylated polymer comprises 4 hydroxyl groups per monomer unit.

In various embodiments, polymers comprise one or more hydroxyl, amine, or thiol groups. The hydroxyl groups, amine groups, or thiol groups are capable of reacting with vinyl sulfone groups of the crosslinker.

In various embodiments, polymers comprise one or more proteins, nucleic acids, lipids, polysaccharides, or carbohydrates.

### Polypeptide and Protein

In various embodiments, the polymer is a polypeptide.

In various embodiments, the polymer is a protein.

In various embodiments, the protein is collagen, zein, silk, silk sericin, silk fibroin, fibrinogen, fibrin, whey, soya, Beta-Lactoglobulin, Casein, Trypsin, Soy Protein (glycinin and β-conglycinin), Gliadin, Albumen, Keratin, Hemoglobin, Gluten, Tropoelastin, or Elastin.

In various embodiments, the protein is natural protein. In various embodiments, the protein is a synthetic protein.

In various embodiments, the protein is an extracellular matrix protein (ECM).

In various embodiments, the ECM is a processed tissue extracellular matrix. As used herein, the term “processed tissue extracellular matrix” means an extracellular matrix (ECM) taken from an animal subject, preferably human, and processed to disinfect and remove cells.

In various embodiments, the polymer comprises one or more of PLA, PHA, PHB, polybutylene succinate, PVA, PHBV, carbonates, PTMC, PDTE, cellulose, modified cellulose, cellulose acetate, HPMC, ethyl cellulose, silk, starch, lignin, acacia gum, pectin, chitin, casein, whey, zein, soya, chitosan, collagen, alginate, keratin, elastin, elastin-like polypeptides, tropoelastin, and hyaluronic acid.

In various embodiments, the protein is a homopolymer of an amino acid, also referred to as a homopoly(amino acid). In various embodiments, the protein is polylysine. In various embodiments, the polylysine is poly-L-lysine (PLL) or poly-D-lysine (PDL). In various embodiments, the protein is polycysteine. In various embodiments, the polycysteine is poly-L-cysteine (PLC) or poly-D-cysteine (PDC). In various embodiments, the protein is poly(γ-glutamic acid) (γ-PGA), poly(ε L lysine) (ε-PL), poly(γ-L-diaminobutanoic acid), or (γ PAB) and poly(L-diaminopropionic acid) (PDAP).

### Oligopeptide

In various embodiments, the polymer is a oligopeptide. In various embodiments, the oligopeptide comprises from 2 to about 50 amino acids. In various embodiments, the oligopeptide comprises from 2 to about 20 amino acids. In various embodiments, the oligopeptide comprises from 2 to about 10 amino acids. In various embodiments, the oligopeptide comprises from 2 to about 5 amino acids. In various embodiments, the oligopeptide comprises from 2 to 3 amino acids. In various embodiments, the oligopeptide comprises 2 acids. In various embodiments, the oligopeptide comprises 3 acids.

In various embodiments, the oligopeptide is RGD.

### Oligosaccharide

In various embodiments, the polymer is an oligosaccharide. In various embodiments, the oligosaccharide is a homopolysaccharide. In various embodiments, the oligosaccharide is a heteropolysaccharide.

In various embodiments, the oligosaccharide comprises from 2 to about 20 monomer units. In various embodiments, the oligosaccharide comprises 2 to about 10 monomer units. In various embodiments, the oligosaccharide comprises 2 to about 5 monomer units. In various embodiments, the oligosaccharide comprises about 3 to about 10 monomer units. In various embodiments, the oligosaccharide comprises about 3 to about 10 monomer units. In various embodiments, the oligosaccharide comprises about 3 to about 5 monomer units. In various embodiments, the oligosaccharide is a disaccharide. In various embodiments, the oligosaccharide is a trisaccharide.

### Polysaccharide

In various embodiments, the polymer is a sugar polymer. In various embodiments, the sugar polymer is a polysaccharide. In various embodiments, the polymer is a polysaccharide. In various embodiments, the polymer is a sugar polymer. In various embodiments, the polysaccharide is a homopolysaccharide. In various embodiments, the polysaccharide is a heteropolysaccharide.

In various embodiments, the polysaccharide is a glycosaminoglycan (GAG).

In various embodiments, the glycosaminoglycan is a heparin or heparan sulfate (HSGAG). In various embodiments, the glycosaminoglycan is a dermatan, dermatan sulfate, chondroitin, or chondroitin sulfate (CSGAG). In various embodiments, the glycosaminoglycan is a keratan or keratan sulfate. In various embodiments, the glycosaminoglycan is a hyaluronic acid.

Examples of polysaccharides include, but are not limited to, sugar-containing molecules such as hyaluronic acid, chondroitin sulfates, keratan sulfates, heparins, amylose, amylopectin, glycogen, cellulose, chitin, callose or laminarin, chrysolaminarin, xylan, arabinoxylan, mannan, fucoidan, inulin, galactogen and galactomannan.

Examples of suitable polymers include, but are not limited to those described in U.S. Pat. No. 10,463,768, which is incorporated herein by reference in its entirety.

In various embodiments, the polymer is a polysaccharide. In various embodiments, the homopolysaccharide is a water-soluble homopolysaccharide. In various embodiments, the homopolysaccharide is polymannuronic acid.

In various embodiments, the polymer is a polyhydroxy acid. In various embodiments, the polyhydroxy acid is luconolactone, galactose, or lactobionic acid.

In various embodiments, the polymer is a polyhexuronic acids. In various embodiments, the hexuronic acid is any uronic acid derived from a hexose. In various embodiments, the hexuronic acid is selected from fructuronic acids, galacturonic acids, glucuronic acids, guluronic acids, iduronic acids, mannuronic acids, and tagaturonic acids.

Examples of polymers include, but are not limited to, hyaluronic acid, hyaluronan, hylan, chondroitin 4-sulfate and chondroitin 6-sulfate, chitosan.

Examples of polymers also include polyanionic polysaccharides, including but not limited to, alkylcarboxy ether derivatives of cellulose and their salts, alginic acid and its salts.

Examples of polymers also include polyhexuronic acids, including but not limited to, pectin, polyglucuronic acid, and polymannuronic acid.

Examples of polymers also include non-charged polysaccharides including but not limited to, starch, glucomannans, galactomannans, pullulan, curdlan, inulin and cellulose and their hydroxyalkyl ether derivatives.

Examples of polymers also include polysaccharide gums, including but not limited to, xanthan, gum arabic, acacia and guar and their alkylcarboxy ether and hydroxyalkyl ether derivatives.

Examples of polymers also include synthetic polymers, including but not limited to, polyvinyl alcohol and polyethyleneimine.

In various embodiments, polymers may be crosslinked to another polymer of different material.

### Hydrogel

In various aspects the polymers form hydrogels.

In various embodiments, the hydrogel comprises hyaluronic acid (HA), polyvinyl alcohol (PVA), polyethylene glycol (PEG), collagen, gelatin, chitosan, fibrin, alginate, agarose, cellulose, or cellulose derivatives.

In various embodiments, the hydrogel materials of the invention can be based on hyaluronic acid (HA) as the hydrogel material. HA is a non-sulfated, linear polysaccharide with repeating disaccharide units which form the hydrogel component. HA is also a non-immunogenic, native component of the extracellular matrix in human tissues, and widely used as a dermal filler in aesthetic and reconstructive procedures.

In certain embodiments, the hydrogel comprises hyaluronic acid (HA). In certain embodiments, the hydrogel comprises carboxymethyl cellulose (CMC). Hydroxyls groups on hydrogels such as HA and CMC allow for simple modification and sterilization by autoclave.

In various embodiments, the hyaluronic acid comprises a high molecular weight HA of about 1 MDa or greater. In various embodiments, the hyaluronic acid comprises a high molecular weight HA of about 1 MDa or less. In various embodiments, the hyaluronic acid comprises a high molecular weight HA of about 500 kDa or greater. In various embodiments, the hyaluronic acid comprises a low molecular weight HA of about 500 kDa or less.

In various embodiments, the hyaluronic acid comprises a molecular weight ranging from about 1 MDa to about 2 MDa. In various embodiments, the hyaluronic acid comprises a molecular weight ranging from about 1 MDa to about 1.5 MDa. In various embodiments, the hyaluronic acid comprises a molecular weight ranging from about 1.5 MDa to about 2 MDa. In various embodiments, the hyaluronic acid comprises a molecular weight ranging from about 500 kDa to about 1 MDa. In various embodiments, the hyaluronic acid comprises a molecular weight ranging from about 500 kDa to about 500 kDa. In various embodiments, the hyaluronic acid comprises a molecular weight ranging from about 100 kDa to about 500 kDa.

In various embodiments, the hyaluronic acid comprises a molecular weight of about 100 kDa. In various embodiments, the hyaluronic acid comprises a molecular weight of about 200 kDa. In various embodiments, the hyaluronic acid comprises a molecular weight of about 300 kDa. In various embodiments, the hyaluronic acid comprises a molecular weight of about 400 kDa. In various embodiments, the hyaluronic acid comprises a molecular weight of about 500 kDa. In various embodiments, the hyaluronic acid comprises a molecular weight of about 600 kDa. In various embodiments, the hyaluronic acid comprises a molecular weight of about 700 kDa. In various embodiments, the hyaluronic acid comprises a molecular weight of about 800 kDa. In various embodiments, the hyaluronic acid comprises a molecular weight of about 900 kDa. In various embodiments, the hyaluronic acid comprises a molecular weight of about 1 MDa. In various embodiments, the hyaluronic acid comprises a molecular weight of about 1.1 MDa. In various embodiments, the hyaluronic acid comprises a molecular weight of about 1.2 MDa. In various embodiments, the hyaluronic acid comprises a molecular weight of about 1.3 MDa. In various embodiments, the hyaluronic acid comprises a molecular weight of about 1.4 MDa. In various embodiments, the hyaluronic acid comprises a molecular weight of about 1.5 MDa. In various embodiments, the hyaluronic acid comprises a molecular weight of about 1.6 MDa. In various embodiments, the hyaluronic acid comprises a molecular weight of about 1.7 MDa. In various embodiments, the hyaluronic acid comprises a molecular weight of about 1.8 MDa. In various embodiments, the hyaluronic acid comprises a molecular weight of about 1.9 MDa. In various embodiments, the hyaluronic acid comprises a molecular weight of about 2 MDa.

### Pores

In various embodiments, the crosslinked polymer forms a scaffold complex with three-dimensional geometry. In various embodiments, the scaffold complex is not generally a uniform solid material. Instead, in various embodiments, the scaffold complexes comprise a plurality of pores present on and/or within a surface of the scaffold complex. The presence, size, distribution, frequency and other parameters of the pores can be modulated during the creation of the scaffold complex.

Mean pore size can be from below about 1 micron to up to above 100 microns, including 1, 2, 3, 4, 5, 6, 7, 8, 9, 10, 15, 20, 30, 40, 50, 60, 70, 80, 90, 100, 150, or 200 microns, and the size thereof may be narrowly tailored, or substantially uniform in size, e.g., such that at least 40%, such as 50%, 60%, 70%, 80%, 90%, 95% or greater than 95% of the pores are in a desired size or within a desired size range.

In various embodiments, pore size ranges from about 0.1 micron to about 200 microns. In various embodiments, pore size ranges from about 1 micron to about 200 microns. In various embodiments, pore size ranges from about 1 micron to about 100 microns. In various embodiments, pore size ranges from about 10 microns to about 100 microns. In various embodiments, pore size ranges from about 1 micron to about 10 microns.

In various embodiments, mean pore size ranges from about 0.1 micron to about 200 microns. In various embodiments, mean pore size ranges from about 1 micron to about 200 microns. In various embodiments, mean pore size ranges from about 1 micron to about 100 microns. In various embodiments, mean pore size ranges from about 10 microns to about 100 microns. In various embodiments, mean pore size ranges from about 1 micron to about 10 microns.

In various embodiments, the mean pore size is about 1, 2, 3, 4, 5, 6, 7, 8, 9, 10, 15, 20, 30, 40, 50, 60, 70, 80, 90, 100, 150, or 200 microns.

In various embodiments, pore size is substantially uniform. In various embodiments, pore size are within a narrow size distribution.

In various embodiments, pores comprise substantially uniform size distribution of at least about 40%, 50%, 60%, 70%, 80%, 90%, or 95% of the pores are within a range of about 100 microns of each other.

In various embodiments, pores comprise substantially uniform size distribution of at least about 40%, 50%, 60%, 70%, 80%, 90%, or 95% of the pores are within a range of about 10 microns of each other.

In various embodiments, pores comprise substantially uniform size distribution of at least about 40%, 50%, 60%, 70%, 80%, 90%, or 95% of the pores are within a range of about 1 micron of each other.

In various embodiments, pores comprise substantially uniform size distribution of at least about 40%, 50%, 60%, 70%, 80%, 90%, or 95% of the pores are within a range of about 100 microns of the mean pore size.

In various embodiments, pores comprise substantially uniform size distribution of at least about 40%, 50%, 60%, 70%, 80%, 90%, or 95% of the pores are within a range of about 10 microns of the mean pore size.

In various embodiments, pores comprise substantially uniform size distribution of at least about 40%, 50%, 60%, 70%, 80%, 90%, or 95% of the pores are within a range of about 1 micron of the mean pore size.

These pores may also be provided in sufficient densities that two or more pores are in contact with each other such that their volumes overlap to form tunnels between them. These tunnels can be mostly free of the composite material forming the scaffold of pores. These tunnels may be mostly circular in shape, or may be more irregular in shape. The shortest distances between these tunnels' edges can be from below about 1 micron to up to 100 microns, including 1, 2, 3, 4 5, 10, 15, 20, 30, 40, 50, 60 70, 80, 90 or 100 microns, and the size thereof may be narrowly tailored, e.g., such that at least 40%, such as 50%, 60%, 70%, 80%, 90%, 95% or greater than 95% of the tunnels are in a desired size or within a desired size range.

### EXAMPLES

Exemplary methods and materials are described below, although methods and materials similar or equivalent to those described herein can also be used in the practice of the present invention and will be apparent to those of skill in the art. The materials, methods, and examples are illustrative only and not intended to be limiting.

### Example 1

### Preparation of DVS-Crosslinked HA Hydrogel

50 μL of 10N sodium hydroxide solution and 85 μL of divinyl sulfone is added to 5 mL of 20 mg/mL HA (MW 1600 kDa, same as other examples unless otherwise noted) solution, to give a final concentration of 0.1N sodium hydroxide, 20 mg/mL HA, and 20 mg/mL DVS. This mixture is incubated at 30° C. for 2 hours to fully gel. After being suspended in 35 mL of 20 mM NaH2PO4 solution for 1 hour under gentle agitation, the gel is injected into a regenerated cellulose dialysis tubing (12-14 kDa cutoff) via a 16-Ga blunt needle and dialyzed against pH 7.2 DPBS. The resulting transparent hydrogels are separated from the dialysis solution using centrifugation and are sterilized via autoclave (at pressure of approximately 15 psi) at 121° C. for 15 minutes.

### Example 2

Preparation of DVS-Crosslinked HA Hydrogel with Quenched Pendant Groups with Various Quenchers

For each group, incubate a 5 ml syringe containing 3.0 ml 27 mg/ml hyaluronic acid (HA) per syringe in a 30° C. water bath for 1 hr. Add 30 ul 10M NaOH to another 5 ml syringe and connect to a syringe containing hydrogel HA through a Luer-lock coupler. Thoroughly mix the contents by pushing back and forth between the syringes for a minute. Using the same method, mix 69 ul divinyl sulfone (DVS) into each pair of syringes (DVS to HA subunit ratio of 3.2 to 1). Then incubate sealed syringe in a 30° C. water bath for 2 hours.

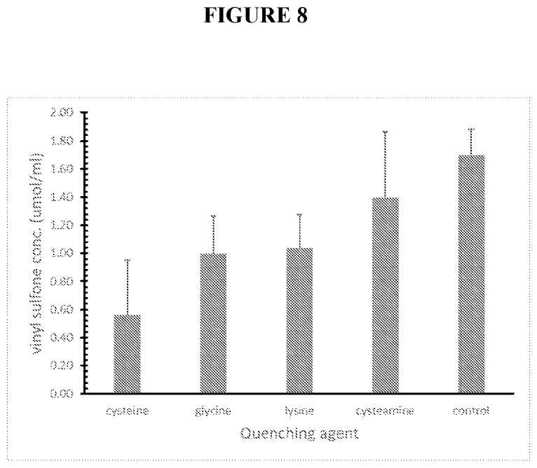

Immediately after the two-hour incubation, the syringes were mixed with a given type of quenching agent. The quenching type's concentrations were normalized to have 1 quenching group for every DVS molecule originally added (or 1 quenching group per every 2 vinyl sulfone groups). The cysteine and cysteamine groups had 2 quenching groups per molecule (1 thiol group and 1 amine group), the lysine group had 2 quenching group per molecule (2 amine groups) and the glycine group had 1 quenching group per molecule (amine group). Therefore, the glycine quenching agent's concentration is double that of the other quenching agents. For each type of quenching agent used, 0.343 ml (cysteine, lysine or cysteamine) or 0.686 ml (glycine) of 1.0 Molar quencher solution is added to one syringe, and then using the luer locking method, is combined with the syringe of DVS-crosslinked HA, and then is mixed for 1 min. The syringe pairs are then incubated at room temperature (approximately 23° C.) for 4 minutes. An unquenched, control group is treated identically, aside from the quenching steps. The quenched gels are then mixed with 22 ml 20 mM NaH₂PO₄ in a 50 ml syringe using the luer locking method to lower the pH and halt further reaction, The resulting syringes are mixed on an orbital shaker at 100 rpm for 1 hr. Then the gel suspensions are injected into dialysis tubing through a 16-gauge blunt needle and dialyzed against DPBS overnight at room temperature (1 L Dialysis volume). The DPBS dialysis solution is then changed with a fresh 1 L of DPBS and dialyzed for a further 3 hrs. The gel in the tubing is then removed from the tubing and centrifuged at 17000×g for 5 min. The supernatant is removed, and the gel is loaded into 1 ml glass syringes. The syringes are terminally sterilized by autoclave (121° C. with at least 15 psi pressure for 30 minute cycle). The groups were then tested for residual vinyl sulfone groups (using assay from Example 6). The results are shown in FIG. 8 (vinyl sulfone concentration in micromoles per mL of gel). The control group has a substantial concentration of vinyl sulfone present, which was reduced by the use of all 4 quenching agent types. The amino-acid quenchers reduced the residuals more effectively than the cysteamine, with the cysteine being the most effective at this concentration.

### Example 3

Preparation of DVS-Crosslinked HA Hydrogel with Cysteine-Quenched Vinyl Sulfone

50 μL of 10N sodium hydroxide solution and 127 μL of divinyl sulfone is added to 5 mL of 30 mg/mL HA (MW 1600 kDa, same as used in other examples unless otherwise noted) solution, to give a final concentration of 0.1N sodium hydroxide, 30 mg/mL HA, and 30 mg/mL DVS. This mixture is incubated at 30° C. for 2 hours to fully gel. The gel (85 μL) is then immediately mixed with 3M cysteine solution and react at ambient room temperature for 5 minutes. The gel is suspended in 35 mL 20 mM monobasic sodium phosphate solution for 1 hour, then the suspension is injected into a regenerated cellulose dialysis tubing (12-14 kDa cutoff) via a 16-Ga blunt needle and then dialyze sequentially against two 1 L volumes of pH 7.2 DPBS. The resulting transparent hydrogels are sterilized at 121° C. for 15 minutes, and then stored at 4° C.

### Example 4

Synthesis of Hyaluronic Acid Hydrogels by Crosslinking HA with Divinyl Sulfone

### Procedure:

1. dissolve 900 mg HA in 30 ml water (30 mg/ml) at 4° C. overnight.
2. aliquot the solution into two 5 ml syringes as 4.5 g per syringe (Lot 1) and (Lot 2).
3. warm up HA solutions to 30° C. in a water bath for 2 hr. and then store at 4° C.
4. add 45 ul 10M NaOH to HA solution to each syringe through a new 5 ml syringe with a luer lock union. Mix well by pushing back and forth between two syringes for a minute.
5. add 115 ul DVS into each syringe containing HA through a new 5 ml syringe with a luer lock union. Mix well by pushing back and forth between the two syringes for a minute.
6. incubate the syringes in a 30° C. water bath for 2 hours.
7. Add 40 ul 300 mM Cys into a new syringe and then combine this with the DVS-crosslinked HA syringes via a luer lock and mix as above for 1 min. Incubate at room temperature for 4 minutes.
8. Remove the empty syringe and connect the HA syringe to a 50 ml syringe containing 35 ml 20 mM NaH2PO4.
9. Push gel's syringe contents into the 50 ml syringe.
10. Hydrate the dialysis tubing at least 15 min before it is to be used.
11. Connect a 16-gauge blunt needle to the syringe and inject its contents into the dialysis tubing.
12. Dialyze gel against 1 L of DPBS for 3 hrs at ambient temperature. Change the dialysis tube to 1 L of fresh DPBS and dialyze overnight.
13. Spin down the dialyzed solution at 17000×g for 5 min to pellet the gel.
14. Aspirate and discard the supernatant.
15. Load the pelleted gel into 1 ml glass syringes.
16. Autoclave the syringes) with liquid cycle (121° C. and 15 PSI) for 15 min.
17. Store the syringes at 4° C.

### Example 5

Synthesis of Hyaluronic Acid Hydrogels by Crosslinking HA with Divinyl Sulfone in Sodium Hydroxide Solution with Quenching Reaction
1. Dissolve 900 mg HA in 30 ml water (30 mg/ml) at 4° C. overnight.
2. Load the solution into 5 ml syringes, 3.5 ml, 4.0 ml, 4.5 ml and 5 ml, respectively.
3. Add water to the syringes via a luer locked second syringe to reach 5 ml total volume, and mix well by pushing back and forth between the two syringes for a minute.
4. Warm up the HA solutions to 30° C. in a water bath for 1 hr.
5. Add 50 ul 10M NaOH to the HA solution through a 5 ml syringe with a union, and mix well by pushing back and forth between the two syringes for a minute.
6. Add DVS (with the amount corresponding to each group in Table 1) into syringes, and mix well by pushing back and forth between two syringes for a minute.
7. Incubate the syringes in a water bath for 2 or 4 hours.
8. Add the corresponding volume of 1.5M Cys stock solution into each syringe and mixed for 1 min. incubate at room temperature for 4 minutes.
9. Remove the empty syringe and connect a syringe containing 35 ml 20 mM NaH₂PO₄.
10. Push 5 ml syringe and transfer all gel to 50 ml syringe.
11. Shake at 100 rpm for 1 hr.
12. Hydrate the dialysis tubing for at least 15 min prior to use.
13. Connect a 16-gauge blunt needle to the syringe and inject into the dialysis tubing.
14. Dialysis gel against 1 L DPBS for 3 hrs. Change to a fresh 1 L of DPBS dialyze over night at ambient temperature.
15. Spin down at 17000×g for 5 min.
16. Remove supernatant and load gel into 1 ml glass syringes.
17. Autoclave syringes with liquid cycle for 30 min (121° C., 15 psi).

TABLE 1

| 27 mg/ml HA (ml) | 10M NaOH (μl) | DVS (μl) | 1.0M Cys (μl) |
|---|---|---|---|
| 3 | 50 | 69 | 343 |
| 3 | 50 | 46 | 229 |
| 3 | 50 | 23 | 114 |
| 3 | 50 | 17 | 86 |

TABLE 2

| | final volume (ml) | final HA conc. (mg/ml) |
|---|---|---|
| Material-1 | 10.8 | 7.5 |
| Material-2 | 15.1 | 5.4 |
| Material-3 | 17.8 | 4.6 |
| Material-4 | 22.0 | 3.7 |

### Example 6

### Ellman-Based Assay for Residual Vinyl Sulfone Functionality

### Buffers:

Ellman's assay buffer: 0.1M Phosphate buffer at pH 8 with 1 mM EDTA.
Reactant Solution: 30 mM Cysteine in Ellman's assay buffer.
DTNB working solution: 80 μg/mL of 5,5′-dithiobis-(2-nitrobenzoic acid) in Ellman's assay buffer.

### Assay:

Add 10 μL of Reactant Solution to 100 μL of each gel sample. Incubate 1 h at 37° C. Add 650 μL of room temperature Ellman's assay buffer to each sample. Centrifuge at 17000×g for 5 min. Mix 20 μL of each supernatant with 200 ul DTNB working solution. After 15 minutes at room temperature, read the samples' absorbance at 412 nm via a spectrophotometer. Determine sample concentration by comparing result to known standards curve or by the known molar extinction coefficient of DTNB (using Ellman's protocols such as provided by Thermo Fisher <https://www.thermofisher.com/document-connect/document-connect.html?url=https://assets.thermofisher.com/TFS-Assets % 2FLSG %2Fmanuals %2FMAN0011216_Ellmans_Reag_UG.pdf>).

### Example 7

### Additional Assays for Analyzing/Detecting Reagents and Products

Fourier Transform Infrared spectroscopy (FTIR) can be used to detect the presence of molecules such as quenchers like cysteine, glycine, or lysine, either in unreacted form or in shifted peaks in reacted form (once reacted with vinyl sulfone), and can measure unbound concentrations (in combination with dialysis prior to analysis) or in bound form (after dissolving gel through methods such as enzymatic or acid/base digestion).

The gel products can also be characterized according to presence of vinyl sulfone to amine or thiol bonding (via methods such as FTIR and NMR).

Gas Chromatography (GC) can be used to measure the concentration of residual unreacted DVS.

Gas Chromatography (GC) can be used to measure the concentration of mono-quenched (1 vinyl sulfone quenched, 1 intact) and double quenched DVS molecules that are not bound to the gel phase.

### Example 8

Preparation of DVS-Crosslinked Hyaluronic Acid and Analysis of Residual Pendant Vinyl Sulfone Groups in Crosslinked HA Gels with Cysteine

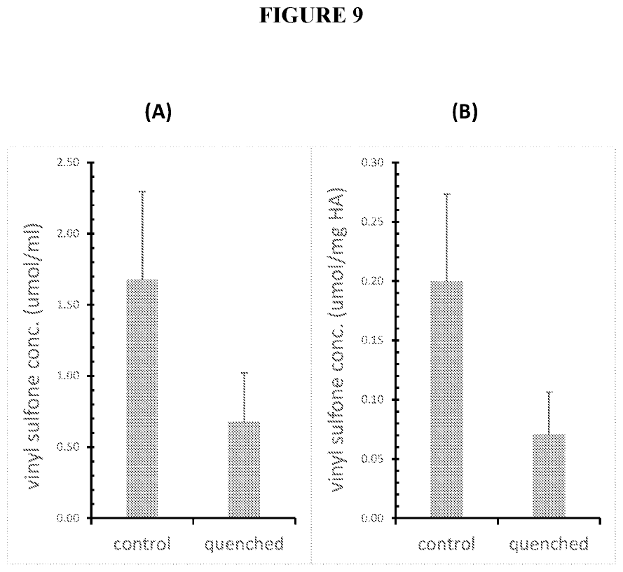

Hyaluronic acid was dissolved in distilled water (4.5 mL at 30 mg/mL), which was made alkaline through the addition of 10N NaOH solution to a final concentration of 0.1M NaOH. DVS (1.146 mMole) was next added under agitation and allowed to react for 2 hr at 30° C. Cysteine (0.012 mMole) was added to the reactant mixture (1:95.5 molar ratio of cysteine to DVS prior to reaction) as a quenching agent. The quencher was reacted with the gel for 5 minutes at 30° C. The control group was prepared identically, with the exception of the addition of the quenching agent. The crosslinked gels were then purified through dialysis as per Example 5. The resulting gels were analyzed for residual pendant vinyl sulfone content through a modified Ellman's assay (see Example 6). Results are shown in FIG. 9. Units of vinyl sulfone concentration are micromoles per mL of gel. The quenching step resulted in a 2.75-fold reduction in the concentration of residual vinyl sulfone groups in the gel (relative to the control group without the quenching step), when measured either versus the total gel volume (part A), or versus the hyaluronic acid content of the gel (part B).

### Example 9

### Residual Vinyl Sulfone Quenching in Complex, Composite Gels.

The improvements of this quenching invention can be applied to more complex gels, such as those with a composite structure of multiphases, such as a fibrous phase dispersed within a hydrogel phase crosslinked with DVS. The fibrous component can react with the vinyl sulfone groups, resulting in both interfacial bonding and residual pendant groups. Such pendant groups may be particularly an issue in such a complex composite if the crosslinker is short (such as DVS) but with long average spacing between molecules due to steric hindrance (wherein one of the DVS functional groups can readily react with a functional group on the fiber or hydrogel component, but the second DVS functional group cannot readily react with a second functional group). This example describes the production of a fibrous component that can be used in such a complex composite, electrospun collagen fibers.

### Collagen Fiber Production:

Lyophilized bovine type I collagen (bovine skin-derived, from Collagen Solutions) was dissolved in 1,1,1,3,3,3-Hexafluoro-2-propanol (HFIP) at room temperature overnight to give a viscous cloudy solution (5.2% w/w). The collagen solution was loaded into 5 mL plastic syringes with a 23G metallic blunt needle. The electrospinning was performed on a Ucalery electrospinning unit with the following parameters: 2.2 mL/h of the flow rate; two syringes in parallel; 17.4 kV of the voltage applied to the needle; 10.8 kv 8 kV (negative) applied to the collector; 12.5 cm of the collecting distance; 200 rpm of the rotation rate of the metallic collector. The resulting collagen nanofibers/microfibers were crosslinked in ethanol containing 50 mM 1-Ethyl-3-(3-dimethylaminopropyl) carbodiimide (EDC) and 20 mM N-Hydroxysuccinimide (NHS) for 20 hours. After the crosslinking, fibers were washed with ethanol three times (30 minutes each) to remove the excessive reagents. The collagen fibers were then broken down into fragments using cryomilling (SPEX SamplePrep).

The amount of intra-crosslinking or inter-crosslinking of collagen fibers may be measured using ¹³C-NMR spectra. (See, A. L. Gatta, et al. “Hyaluronan Hydrogels: Rheology and Stability in Relation to the Type/Level of Biopolymer Chemical Modification,” Polymers 2022, 14, 2402.) The ratio of uncrosslinked to crosslinked collagen can be measured with the method in example #5.

### Example 10

### Preparation of HA Hydrogels and NHCs Crosslinked by DVS:

Test lots of a series of gels were produced, with and without a fibrous phase, with a lot size of 3cc (during gelation stage). Stock solutions of the reagents were prepared. Sodium hyaluronate (HA, MW 1.5 MDa) was dissolved in distilled water at a concentration of 30 mg/mL. Sodium hydroxide was dissolved in water to make a 10M solution. Lysine monohydrochloride was dissolved in water at a concentration of 2M. Firstly, gels without collagen were produced with a gelation HA concentration of 21,24,27, or 30 mg/mL and a DVS-to-HA subunit ratio of 3.22, 3.5, 4, 4.5, or 5×, by varying the amount of each stock solution added, with 0.03 mL of the sodium hydroxide stock added to achieve the targeted 0.1M concentration. Each group was mixed between two 5 mL syringes connected with a luer-lok connector. After vigorous mixing, the mixture was incubated in sealed syringes in a 30° C. water bath for 2 hours. The reaction was chemically quenched by lysine solution (molar ratio of 1 to 1 of lysine to DVS molecules), with syringe-syringe mixing followed by a 5-minute incubation at room temperature. The gels are then neutralized in 50 mL syringes containing 21 mL 20 mM monobasic sodium phosphate solution. The hydrogel was transferred into dialysis tubing (13,000 MWCO) and dialyzed against 1 L 0.9% sodium chloride for 16 hours and 1 L DPBS for 3 hours at room temperature. The resulting gel suspension was transferred to 1.5 mL microcentrifuge tubes through 18-gauge blunt needles then centrifuged at 17,000×g for 3 minutes to separate excess water. After removing the supernatant, the gels were collected in 10 mL syringes and weighed in order to calculate the swelling ratio (relative to weight of initial gelation solution, see example #5). The gels were beaded through 150-micron screens, then loaded into 1 mL glass syringes and autoclaved at 121° C. for 30 minutes. The swelling ratio of the gels during manufacturing and the resulting final HA concentrations are plotted in Figures #10-13.

Secondly, gels were formulated with the same methodology, but additionally with gelatin fibers (porcine, purchased from Sigma) or collagen fibers (bovine, from Collagen Solutions) prepared via the method of example 1. The collagen fiber concentration in the pre-gel solution varied from 37 to 70 mg/mL, the pre-gel HA concentration was set to 21,23, or 25 mg/mL, and the DVS-to-HA subunit ratio was varied from 2.3 to 4.5×. Each group was mixed between two 5 mL syringes connected with a luer-lok connector. After vigorous mixing, the mixture was incubated in sealed syringes in a 30° C. water bath for 2 hours. The reaction was chemically quenched by lysine solution (molar ratio of 1 to 1 of lysine to DVS molecules), with syringe-syringe mixing followed by a 5-minute incubation at room temperature. The gels are then neutralized in 50 mL syringes containing 21 mL 20 mM monobasic sodium phosphate solution. The composite hydrogel was transferred into dialysis tubing (13,000 MWCO) and dialyzed against 1 L 0.9% sodium chloride for 16 hours and 1 L DPBS for 3 hours at room temperature. The resulting gel suspension was transferred to 1.5 mL microcentrifuge tubes through 18-gauge blunt needles then centrifuged at 17,000×g for 3 minutes to separate excess water. After removing the supernatant, the gels were collected in 10 mL syringes and weighed in order to calculate the swelling ratio (relative to weight of initial gelation solution, see example #5). The composite gels were beaded through 150-micron screens, then loaded into 1 mL glass syringes and autoclaved at 121° C. for 30 minutes.

### Example 11

### Mechanical Properties of Hydrogels and NHCs:

The rheological characterizations of hydrogels and composite gels were performed as described previously. Rheometry (AR2000ex, TA Instruments) was harnessed to measure various shear mechanical properties of hydrogels and composite gels using a parallel plate geometry of 25 mm with 0.9 mm gap at 25° C. A linear viscoelastic region was measured for the gels by a strain sweep with an increasing shear strain amplitude at a set frequency (1 Hz). The three main properties were storage modulus (G′), loss modulus (G″) and tan delta (G″/G′). The injection forces of bulk gels and microgels of HA and NHC were measured by extruding 1 mL sample through a 27-gauge needle in 1 mL BD syringe using Instron load frame (34SC-05, Norwood, MA).

### Example 12

### Preparation of DVS Crosslinked Collagen-HA Composite Groups for Animal Study

Sodium hyaluronate (HA, MW 1.5 MDa) was dissolved in distilled water at a concentration of 30 mg/mL. Sodium hydroxide was dissolved in water to make a 10M solution. Lysine monohydrochloride was dissolved in water at a concentration of 2M.

TABLE 3

| | HA | water | | | | |
|---|---|---|---|---|---|---|
| Group | solution(mL) | (mL) | fiber(g) | NaOH(mL) | DVS(mL) | lysine(mL) |
| M1 | 5.00 | 1.00 | 0.270 | 0.060 | 0.159 | 0.792 |
| M2 | 5.00 | 1.00 | 0.270 | 0.060 | 0.139 | 0.692 |
| M3 | 4.98 | 1.52 | 0.422 | 0.065 | 0.178 | 0.887 |
| M4 | 4.60 | 1.40 | 0.390 | 0.060 | 0.146 | 0.727 |

According to Table 3, for each group, the HA stock solution volume specified in above table was blended with the specified amount of water, collagen fibers and sodium hydroxide solution in two 10 mL syringes connected with a luer-lok connector for syringe-syringe mixing. The specified DVS volume is then added to each group and mixed via-syringe-syringe mixing. After vigorous mixing, the mixture was incubated in sealed syringes in a 30° C. water bath for 2 hours. The reaction was chemically quenched by lysine solution (molar ratio of 1 to 1 of lysine to DVS molecules), with syringe-syringe mixing followed by a 5 minute incubation at room temperature. The gels are then neutralized in 60 mL syringes containing 42 mL 20 mM monobasic sodium phosphate solution. The resulting hydrogel suspension was transferred into dialysis tubing (13,000 MWCO) and dialyzed against IL DPBS 3 times (8 to 16 hours each time, room temperature) then particularized with 150 μm stainless steel wire cloth discs in 25 mm filter holders. The resulting gel suspension was centrifuged at 4,000×g for 10 minutes to separate excess water. After removing the supernatant, the gel composites were weighed in order to calculate the swelling ratio (relative to weight of initial gelation solution). The swelling ratio was then verified by directly measuring the hyaluronic acid content and the collagen content of the final gel. The gel composite was loaded into 1 mL glass syringes then autoclaved at 121° C. for 30 minutes.

### Example 13

### Measurement of Component Concentrations in Gel Composite

### Carbazole Assay for Hyaluronic Acid Content in Gel Composite

The concentration of hyaluronic acid in the final gel sample can be calculated through a carbazole-based assay. Briefly, 3.00 mL 0.025M sodium tetraborate in sulfuric acid was added into a 7-mL glass tube containing 0.030-0.040 g gel composite sample and heated in a boiling water bath for 15 min before being cooled down at room temperature for 15 minutes. Then, 0.80 mL 0.125% carbazole in absolute ethanol (w/w) was added into each tube and heated in boiling water bath for 15 min. After being cooled down at room temperature for 15 minutes, 100 μL of final solution was transferred to a 96-well plate and read at 530 nm on a microplate reader (Epoch; BioTek). The Hyaluronic acid content of the gel was calculated from a standards curve (generated via identical testing of a known-concentration hyaluronic acid solution).

### Sirus Red Adsorption Assay for Collagen Fiber Content in Gel Composite

The concentration of collagen in the final gel sample can be calculated through a colorimetric assay. Briefly, 10.00 ml 1 mg/ml Sirius red F3B solution in 1:10 (v:v) acetic acid was added to a 15 mL centrifuge tube containing 0.080-0.100 g gel composite and vortex at maximum speed for 1 minute. The sample was covered with aluminum foil and shaken on an orbital shaker at 100 rpm for 5 hours at room temperature. 1 mL solution was transferred into a microcentrifuge tube and spin down at 10,000×g for 3 minutes. 0.05 ml of supernatant was diluted into a microcentrifuge tube containing 1 mL of water. 0.2 ml diluted solution was put into a 96-well plate to read at 530 nm. A series of samples of known amounts of collagen (0.2-2 mg) were used to make a standard curve that was used to calculate the concentration of the test sample.

### Free (Uncrosslinked) Collagen in Collagen Fiber

1.00 mL water was added to 1.5 mL microcentrifuge tubes containing 0.020-0.025 g collagen fiber. The tubes were agitated on a vortex mixer at maximum speed for 1 minute, followed by incubation on an orbital shaker (100 rpm) at room temperature for 4 hours. Fiber was spun down at 17,000×g for 3 minutes and 0.1 mL supernatant was mixed with 0.8 mL BCA working solution (Thermo Scientific), then incubated at 37° C. for 30 minutes. When the samples were cooled down to room temperature. 0.2 mL was transferred to a 96-well plate and the absorbance was read at 562 nm on an Epoch microplate spectrophotometer (Bio Tek instruments). Bovine serum albumin (Thermo Scientific) was used as standards. The free collagen content in collagen fiber was determined as 3.1±0.05%.

### Free (Uncrosslinked) HA in Matrix-C Gel Composite

1.00 mL water was added to the centrifuge tubes containing 0.500 g Matrix-C samples. The tubes were agitated on a vortex mixer at maximum speed for 1 minute, followed by incubation on an orbital shaker (100 rpm) at room temperature for 2 hours. Samples were spun down at 17,000×g for 3 minutes. 0.050 mL was added to 0.500 mL 0.005% Stains-All in 55% DMSO, followed by mixing on a vortex mixer for 1 minute. 0.100 mL sample was transferred to a 96-well plate and read at 655 nm. Sodium Hyaluronate (LifeCore) was used as a reference material. Free HA content in M1 and M2 were ranging from 5.6-5.9%.

### Example 14

### In Vivo Animal Study

Four treatment groups were prepared in Example #4 from the same batch of raw materials and are manufactured to be as similar as possible aside from the experimental variables (pre-gelation concentrations of the HA, Collagen fiber, and crosslinker (DVS) concentration). Collagen fibers were produced from Collagen Solutions Type I bovine collagen as per example #1. The Hyaluronic Acid was purchased from LifeCore Biomedical Corporation. The DVS was purchased from TCI (Tokyo Chemical Industry). At the end of gelation, the residual sulfone groups are chemically quenched with a lysine addition (Sigma).

### Test Groups

The four test groups have varying levels of collagen fiber loading and crosslinking to assess the effects of those variables on the biologic response. Each group used 1.6 MDa Hyaluronic acid.
Group M1: Gel produced with the gelation conditions of 25 mg/mL HA, 45 mg/mL Collagen, and a [DVS]/[HA Repeat Unit] ratio of 4.0:1 (264 mM of DVS). The resulting gel has final gel concentrations of approximately 9.7 mg/mL of HA and 17.5 mg/mL of collagen in DPBS with a stiffness (G′) of 628 Pa and an injection force of 6.1N.
Group M2: Gel produced with the gelation conditions of 25 mg/mL HA, 45 mg/mL Collagen, and a [DVS]/[HA Repeat Unit] ratio of 3.5:1 (231 mM of DVS). The resulting gel has final gel concentrations of approximately 8.7 mg/mL of HA and 15.7 mg/mL of collagen in DPBS with a stiffness (G′) of 534 Pa and an injection force of 7.8N.
Group M3: Gel produced with the gelation conditions of 23 mg/mL HA, 65 mg/mL Collagen, and a [DVS]/[HA Repeat Unit] ratio of 4.5:1 (273 mM of DVS). The resulting gel has final gel concentrations of approximately 10.2 mg/mL of HA and 27.5 mg/ml of collagen in DPBS with a stiffness (G′) of 610 Pa and an injection force of 4.9N.
Group M4: Gel produced with the gelation conditions of 23 mg/mL HA, 65 mg/mL Collagen, and a [DVS]/[HA Repeat Unit] ratio of 4.0:1 (242 mM of DVS). The resulting gel has final gel concentrations of approximately 9.5 mg/mL of HA and 25.1 mg/mL of collagen in DPBS with a stiffness (G′) of 548 Pa and an injection force of 5.2N.

Control Group C: Voluma XC, a commercial market-leading soft tissue filler comprised of crosslinked hyaluronic acid.

### Test System

Species: Rat
Breed: Sprague Dawley
Source: Single USDA licensed supplier
Sex: Female
Age: Young Adult
Number of Animals: 27
(4 rats per test group timepoint and 6 timepoints, with 3 control rats)
Identification Method: Ear tag or marking.

### Injection Procedure

The rats are anesthetized with isoflurane and their backs are shaved with clippers. The back is then disinfected (Iodine) immediately before injection.

At each injection site, 200 ul injected as a single, round bolus subcutaneously in dorsal region of rat with 27G×½″ needle. Each injection bolus has a margin of at least 5 mm from each other bolus.

Site Location Map: the injections will be at the back of the rat per the implant scheme below:

### Implant Scheme:

### Test Groups

Cranial

$$( \begin{matrix}T 1 \\ T 2\end{matrix} \vert \begin{matrix}T 3 \\ T 4\end{matrix} )$$

Caudal
T1=Test Group 1, T2=Test Group 2, T3=Test Group 3, T4=Test Group 4

### Control Group:

Cranial

$$( \begin{matrix}C \\ C\end{matrix} \vert \begin{matrix}C \\ C\end{matrix} )$$

Caudal
C=Control group (Voluma XC)

### Sample Size

Test groups: 4 replicates per group per timepoint, each replicate coming from a separate animal. (4 test group rats per timepoint, for a total of 16 test sites combined per timepoint).
Control: 4 replicates per timepoint, in the same animal.

### Live Assessment Timepoints

The Animals will be scored for erythema and edema immediately after injection, and 4 days after injection. They will be graded on the scale from ISO 10993-10 table 3.

MRI Timepoints: 0 days, 1 day, 1 week, 4 weeks, 8 weeks, 26 weeks, 55 weeks, 69 weeks.

### Tissue Harvest Timepoints

The timepoints for this study were 0 day, 1 day, 1 Week, 4 weeks, 8 weeks, 20 weeks, 26 weeks, 40 weeks, and 69 weeks. The last timepoints are assigned based upon the evaluation at the 26-week timepoint. Unless otherwise noted, the 1 week timepoint is tested exactly 7 days after the procedure and the 4-week timepoint will be tested within 2 days of the planned timepoint. The 8 week and beyond test groups will be tested within 7 days of the planned timepoint. 1 rat of the control group will be harvested at each timepoint.

The rats from group 2 will be imaged at every timepoint. Additionally, at each harvest timepoint, the rats-to-be-harvested may be imaged prior to tissue harvesting. The timepoints correspond to 0 days (establishing baseline immediately after injection) 1 day post injection (to assess swelling), and at designated tissue harvest timepoints.

### Tissue Harvest Timepoints

Planned timepoints for this study are 1 Week, 4 weeks, 8 weeks, 26 weeks, with two TBD timepoints. The first TBD timepoint will be assigned based upon the evaluation at the 26-week timepoint and the second TBD timepoint will be assigned based upon the evaluation of the TBD 1 timepoint. The 1 week timepoint will be tested exactly 7 days after the procedure and the 4-week timepoint will be tested within 2 days of the planned timepoint. The 8 week and beyond test groups will be tested within 7 days of the planned timepoint. 1 rat of the control group will be harvested at the 26-week timepoint and at each of the TBD timepoints.

Euthanasia. After any MRI imaging, the designated rats will be humanely euthanized. The dorsal skin is dissected open such that all four injection sites can be visually identified. The test and control sites are excised as full tissue blocks and fixed in formalin overnight, then processed for histological and immunofluorescent assessments.

### Acceptance Criteria

Swelling: Less than 200% initial volume (which is the POD 0 measurement, expected to be approximately 200 μL) and less than commercial control (Voluma XC) at first measurement (1 day post-injection, POD 1).
Edema: Mean scores less than or equal to the mean scores for Voluma XC per rubric of Table 3 of ISO-10993-10 at POD 4
Irritation: Erythema mean scores less than or equal to the mean scores for Voluma XC per Table 3 of ISO 10993-10 (See attachments) at POD 4
Degradation: Degradation will be considered to be complete when all viewed sites score 4 on the ISO 10993-6 degradation scale (as interpreted by NAMSA and LifeSprout for Lumina, see attachment 2 below). First timepoint with all 4's (when analyzing at least 3 sites) will be considered time of complete degradation.
Volumization: The mean implant volume will be measured for each group at each MRI timepoint. Each test group will be considered to have acceptable volumization properties if the mean measured volume is greater than 50% of original (PODO) volume at each timepoint through 26 weeks.
Cellular Infiltration Rate: Cell infiltration rate is calculated by the percentage area covered by the infiltrated cells inside the injected materials. The acceptance criteria for cellular infiltration rate is at least 50% increase of the average cell infiltration rate compared with control (Voluma XC) at 26 week timepoint.
Cellular Density: Cell density is counted based on the immunofluorescence images stained with DAPI (staining individual cell nucleus). The acceptance criteria of cellular density is at least 50% higher mean cell density inside the injected implant compared with
Angiogenesis: The measurements of angiogenesis will be based on the immunofluorescence images where tissue slides are stained with RECA-1 (endothelial cells) and α-SMA (pericytes). The criteria for endothelial cells will be the distance infiltrating from the periphery to the middle, and that for pericyte will be the densities per unit area (#/mm\hat{ }2).

Cell type, adipogenesis, macrophage polarization: Adipogenesis and macrophage polarization measurements will be based on immunofluorescence imaging. Perilipin-1 is used to stain the adipocytes forming inside the materials, while CD68, CD38 and CD163 are used to stain the macrophage phenotypes inside the materials. For both adipogenesis and macrophage polarization, cell density will be a method to quantitatively compare the test groups with the Voluma control.

TABLE 4
Grading system for intracutaneous (intradermal) reactions

| | Numerical |
|---|---|
| | Grading |
| Erythema and eschar formation | |
| No erythema 0 | 0 |
| Very slight erythema (barely perceptible) | 1 |
| Well-defined erythema | 2 |
| Moderate erythema | 3 |
| Severe erythema (beet-redness) to, eschar formation | 4 |
| preventing grading of erythema | |
| Oedema formation | |
| No oedema | 0 |
| Very slight oedema (barely perceptible) | 1 |
| Well-defined oedema (edges of area well-defined by definite | 2 |
| raising) | |
| Moderate oedema (raised approximately 1 mm) | 3 |
| Severe oedema (raised more than 1 mm and extending beyond | 4 |
| exposure area) | |
| Maximal possible score irritation | 8 |

### Results:

### Live Assessments:

All 4 treatment groups were well tolerated by the animals, without inducing infection, necrosis, or gross inflammation in the live animals. The implants remained as persistent bumps protruding from the skin at the same location as originally injected. At POD4 (post-operative day 4), the animals were inspected and scored for erythema and oedema per the grading system from ISO 10993-10. All 4 groups met the acceptance criteria described in the protocol. The erythema score were all mild, and all below the score of Voluma XC. The Oedema scores were also all below that of Voluma. This indicates that the formulations are low irritation and low swelling, making them excellent candidates for dermal applications.

Table 5. Mean Erythema and Oedema scoring of prototypes and commercial control at post-operative day 4, scored per rubric in Table 4. All treatment groups met their acceptance criteria, with substantially lower erythema and oedema results than Voluma XC, indicating excellent biocompatibility.

TABLE 5

| Group | Average Erythema | Average Oedema |
|---|---|---|
| M1 | 0.00 | 2.08 |
| M2 | 0.00 | 2.25 |
| M3 | 0.08 | 2.42 |
| M4 | 0.17 | 2.75 |
| Voluma XC | 0.42 | 3.75 |

### Explantation:

During explantation, the implantation sites were photographed intact. The injected boluses of material had remained in place, and maintained a bolus shape. The color of all implant sites (all 4 groups, all replicates, at days 7, 30, and 63) was white to yellow, remarkably similar to native fat tissue. Blood vessels can be discerned either on or near the boluses. The boluses are distinct from pre-existing tissue structures.

### MRI Volume Measurement

The 4 groups showed little to no post-procedural swelling at days 1 and 7, maintaining their volume as intended.

Table 6 shows the quantification of the swelling via MRI at day 1 for the test formulations M1-M4 of example #6 one day after injection. The swelling % is the percentage of additional volume measured at day 1 over day 0. All 4 formulations had 20% swelling or less, while the Voluma XC had 100% swelling (doubled in volume).

TABLE 6

| Group | Day 1 Swelling % | std dev |
|---|---|---|
| M1 | 14% | 15% |
| M2 | 20% | 30% |
| M3 | 12% | 38% |
| M4 | 16% | 22% |
| Voluma XC | 100% | 19% |

The mean swelling percentage for the treatment groups was 12-20%. This is in contrast to Voluma, which doubled in size due to swelling by day 1 (100% swelling), which persisted through day 7 (and then slowly decreased through day 63). This shows a variant of the Matrix-C gel can be made without undesired swelling. The 4 groups and Voluma had broadly similar volumization profiles through day 63, with no-to-slight declines from the day 1 values. The Voluma group had the most absolute volume due to the extensive initial swelling. Normalizing to the day 1 volumes, the groups all maintained 75% or more of the original volume through 63 days. Group M1 had the best volume maintenance, retaining 97% of its day 1 volume at day 63, virtually indistinguishable from Voluma's 94% volume maintenance.

### Histological Assessment:

The gel was clearly discernable in the subcutaneous space, as a light blue gel with dark blue-to-purple fibers. The gel samples remained as coherent boluses at the injection site, and groups M3 and M4 were most resistant to any spreading, with a high gel thickness at all timepoints. The gel groups integrated well with the surrounding tissue, without necrosis or encapsulation. In groups M1 and M2, bands of cellular ingrowth were apparent at the POD7 timepoint, and were extensive in the POD 30 and 63 timepoints, with a plurality of blood vessels formed within the material together with light collagen deposition. Cellular ingrowth and angiogenesis was also seen in groups M3 and M4, but was less extensive, with large sections still acellular at the day 63 timepoint. At the 63 day timepoint, active adipogenesis appears to be occurring in the M1 group in particular, with vascularized adipose tissue at the periphery of the gel bolus and globular, adipocyte-like structures forming within the neighboring gel. The Voluma group appeared to be biocompatible, without necrosis or inflammation, with a thin collagenous band around the periphery of the gel bolus, but without cellular migration into the gel, nor blood vessel growth, or adipose tissue generation.

Through day 63, the matrix-C prototypes are excellent candidates for dermal filler applications, and compare favorably to a commercial leader, Juvederm Voluma XC. In particular, Group M1 had a better irritation and swelling profiles than Voluma, equally maintained the injection volume through 63 days, yet additionally induces extensive cellular integration, and angiogenesis without fibrosis or encapsulation. At the day 63 timepoint, the histology slides indicate that active adipogenesis is underway in the M1 gel, which is generating new, healthy-looking fat in replacement of the gel. This indicates that M1 can be injected safely, with no swelling and predictable response, and maintaining the volumization even as it is turned into vascularized, healthy fat.

### 20 & 26 Week Timepoints

At the 20 week timepoint, the adipose pockets in the panniculus carnosus layer have grown extensively in the M1 group. A large vascularized adipose tissue greater than 1 mm in length and width can be seen within the center of the M1 gel in one example.

The groups M3 and M4 gels exhibited cellular ingrowth rates slower than that of groups M1 and M2.

The Voluma XC group still does not show cellular ingrowth into the gel. There is some small areas of adipose tissue in surrounding tissue, but none within the gel or panniculus carnosus near the gel. The gel has been encapsulated in a thin band of cells and collagen.

### 40 Week Histology

At 40 weeks, the same trend continues. Depending upon the slice location within the implant, the gel has been partially replaced by adipose tissue. The groups M1 and M2 had substantially more cellular ingrowth and remodeling than groups M3 and M4, demonstrating the sensitivity of the body's response to the gel formulation, especially the concentration of collagen fibers and crosslinking agent, DVS.

### 69 Week Histological Assessment

The animal study has been terminated with the final timepoint group harvested at the 69-week timepoint (16 months) due to the age and size of the animals (too large to be imaged alive in the MRI apparatus). The implant sites were harvested en bloc with the surrounding skin tissues and immediately imaged ex vivo in the MRI machine to get final volumization data, before being prepared for histological assessment. The gels are still visibly present at the implant site and are well tolerated, with no signs of localized irritation or fibrosis. Histological assessments have yet to be completed on these tissues.

Differential adipogenesis in or near panniculus carnosus layer in matrix-c prototypes compared to Voluma XC.

In the histological slides, there is enhanced adipogenesis seen in the panniculus carnosus layer of the rat skin compared to that seen in the Voluma XC group. This is visible in either enlarged regions of vascularized adipose tissue immediately above or below the muscle layer when near the implanted gel, or in a greatly increased number of adipocytes in the pockets within the panniculus carnosus layer that typically surround nerves. This adipocyte generation in the pockets of the panniculus carnosus neighboring the implanted gel site is also seen in sections cut longitudinally to the panniculus carnosus muscle layer. This shows that the adipocyte structures are not a sectioning artifact. Without being bound by theory, the adipogenesis may be directly the result of gel that flowed into the pocket during injection, or indirectly caused by the biological signals from the cells directly interacting with the nearby gel. Potential precursors for the adipocytes generated include pericytes from the vasculature and mesenchymal stem cells or fibro-adipogenic progenitors from the muscle. This muscle layer is not a major feature of human skin, unlike in the rat model, but this indicates that the Matrix-C test articles may be more capable of inducing targeted adipogenesis than Voluma XC.

### SUMMARY

All 4 prototype gel groups were well-tolerated, with minimal-to-no acute adverse response, with an erythema and oedema profile superior to that of Voluma XC. No adverse events were noted for any of the rats with the M1-M4 test groups over the entirety of the long term study. The test gels maintained volume with little-to-no swelling in the initial days after injection (days 1 and 7), compared to Voluma XC, which doubled in volume. From Day 7 onwards, the gel groups maintained volume, with less volume loss than the Voluma XC control through 385 days, with a flatter slope in both plots of absolute volume per time and relative volume per time. While still maintaining volumization, the gels also showed marked cellular infiltration, with cells growing into the implanted gel with natural dendritic cell morphology by day 7. The cells grew progressively into the gel over the course of the study, replacing the implanted material with vascularized, cellular tissue complete with diffuse collagen. Potential preadipocytes can be seen on the periphery of early timepoints, and extensive pools of vascularized adipose tissue can be seen intermingled with the remaining gel in the M1 and M2 test groups by 9.5 months. Adipocytes are only seen on the periphery of the gels in groups M3 and M4, and only in the surrounding tissue for the Voluma XC control group. This volumization maintenance together with cellular ingrowth demonstrates the optimal balance between gel degradation rate and the replacement by cells and new tissue. The extent of cellular infiltration varied greatly from group to group, demonstrating the effectiveness and importance of the specific formulation choices for each test group.

The M1 group maintained 85% of its initial volume through more than a year and generated vascularized adipose tissue, making it an excellent candidate for dermal filling applications and body sculpting.

The M2 group exhibited more gel spreading for more a diffuse effect, as might be wanted for superficial placement in the body. The lower crosslinking and collagen concentrations and resulting larger pore size allows for more rapid cellular movement and diffusion for therapeutic indications for cell or drug delivery or increasing cell homing and interactions within the implanted gel.

The M3 and M4 groups had much slower cellular infiltration, and maintained the original shape, resisting spreading. These formulation groups are good candidates for use where implant traits like slow degradation, geometric stability are desired. Examples include implantation of the device into high tissue tension regions such as supraperiosteal placement in the body or weight-bearing locations, or for indications where a more permanent plug is desired, such as for fistula repair, hernia repair, coating permanent implants, etc.

An ideal soft tissue filler would initially immediately fill the defect site with 100% of the target volume, with similar mechanical properties to the surrounding soft tissue, without migration or flow of the filler, and without inducing pain or irritation. The filler should not induce swelling, and then should maintain nearly 100% of its volume indefinitely, while paradoxically degrading away completely without causing scarring or adverse events (such as nodules or granulomas). The test formulations come much closer to achieving this “ideal” profile than any current products such as the Voluma XC control included in this study.

As additional evidence, the test gels induce a large increase in adipocytes in the panniculus carnosus, the striated cutaneous muscle layer in many lower mammals such as rats. The panniculus carnosus includes periodic pockets of nerves surrounded by collagen, blood vessels, and occasionally a small number of adipocytes. The pockets in the panniculus carnosus layer of the Voluma XC control are Without being bound by theory, the adipogenesis may be directly the result of gel that flowed into the pocket during injection (gel can be seen in some pockets at early timepoints), or indirectly caused by the biological signals from the nearby gel, either from cells directly interacting with the nearby gel or gel degradation products.

1. A method of manufacturing a crosslinked polymer comprising steps of:
(A) reacting a polymer material and crosslinking agent, wherein the crosslinking agent comprises two or more vinyl sulfone groups,
whereby the polymer is crosslinked by the vinyl sulfone groups, and
whereby the crosslinked polymer comprises a pendant vinyl sulfone group; and
(B) reacting the pendant vinyl sulfone group with a quenching agent;
whereby the pendant vinyl sulfone group is quenched.

2. The method of claim 1, wherein the crosslinking agent comprises a DVS, a PEG-VS, a PEG-DVS, a PEG-tri-VS, or a PEG-tetra-VS.

3. (canceled)

4. (canceled)

5. (canceled)

6. (canceled)

7. The method of claim 1, wherein the method results in a reduction of residual vinyl sulfone groups.

8. The method of claim 1, wherein the method results in a reduction of residual vinyl sulfone groups:
i. by a range of about 10% to about 90%;
ii. by at least 10% at least 18%, at least 20%, at least 50%, at least 60%, or at least 67%, or at least 50%.

9. (canceled)

10. (canceled)

11. (canceled)

12. (canceled)

13. (canceled)

14. (canceled)

15. The method of claim 1, wherein the crosslinked polymer comprises a hydrogel.

16. The method of claim 15, wherein the hydrogel comprises a reduced number of pendant vinyl sulfone groups.

17. The method of claim 15, wherein the hydrogel comprises residual sulfone groups at a concentration:
i. of at most about 2.0 μmol/mL hydrogel, at most about 1.7 μmol/mL hydrogel, at most about 1.7 μmol/mL hydrogel, at most about 1.4 μmol/mL hydrogel, at most about 1.0 μmol/mL hydrogel, or at most about 0.5 μmol/mL hydrogel; or
ii. ranging from about 0.5 μmol/mL to about 1.4 μmol/mL hydrogel.

18. (canceled)

19. (canceled)

20. (canceled)

21. (canceled)

22. (canceled)

23. The method of claim 1, wherein the hydrogel comprises hyaluronic acid.

24. The method of claim 23, wherein the hyaluronic acid is present:
i. in an amount ranging from about 5 mg/mL to about 15 mg/mL; an amount ranging from about 7 mg/mL to about 12 mg/mL, an amount ranging from about 7 mg/mL to about 10 mg/mL, or an amount ranging from about 9 mg/mL to about 10 mg/mL;
ii. in an amount of about 9 mg/mL, about 9.5 mg/mL, or about 10 mg/mL.

25. (canceled)

26. (canceled)

27. (canceled)

28. (canceled)

29. (canceled)

30. (canceled)

31. The method of claim 23, wherein the hyaluronic acid is present at gelation;
i. in an amount ranging from about 20 mg/mL to about 30 mg/mL; an amount ranging from about 20 mg/mL to about 25 mg/mL, or an amount ranging from about 23 mg/mL to about 25 mg/mL; or
ii. in an amount of about 23 mg/mL, or about 25 mg/mL.

32. (canceled)

33. (canceled)

34. (canceled)

35. (canceled)

36. The method of claim 2, wherein the divinyl sulfone is present for crosslinking:
i. in an amount ranging from about 1:1 to about 5:1, an amount ranging from about 2:1 to about 5:1, an amount ranging from about 3:1 to about 5:1, or an amount ranging from about 3:1 to about 5:1 divinyl sulfone molecules per hyaluronic acid subunit; or
ii. in an amount of about 3.5:1, an amount of about 4:1, or an amount of about 4.5:1 divinyl sulfone molecules per hyaluronic acid subunit.

37. (canceled)

38. (canceled)

39. (canceled)

40. (canceled)

41. (canceled)

42. (canceled)

43. The method of claim 2, wherein the PEG-DVS is present for crosslinking:
i. in an amount ranging from about of 1 mg/mL to about 10 mg/mL, in an amount ranging from about of 1 mg/mL to about 5 mg/mL, in an amount ranging from about of 3 mg/mL to about 10 mg/mL, or in an amount ranging from about of 3 mg/mL to about 5 mg/mL; or
ii. in an amount of about 3 mg/mL, about 5 mg/mL, or about 10 mg/mL.

44. (canceled)

45. (canceled)

46. (canceled)

47. (canceled)

48. (canceled)

49. (canceled)

50. The method of claim 1, wherein the crosslinked polymer comprises a hydrogel network, and the polymer material comprises:
i. a hydroxylated polymer;
ii. a hyaluronic acid material; or
iii. a polysaccharide.

51. (canceled)

52. (canceled)

53. The method of claim 1, wherein the crosslinking agent is a divinyl sulfone.

54. The method of claim 50, wherein:
i. the crosslinking agent comprises a divinyl sulfone; or
ii. the hydroxylated polymer comprises a hyaluronic acid material a divinyl sulfone.

55. (canceled)

56. A hydrogel product by the process comprising the steps of:
(A) reacting a hyaluronic acid material and a crosslinking agent, wherein the crosslinking agent comprises two or more vinyl sulfone groups,
whereby the hyaluronic acid is covalently crosslinked by the vinyl sulfone groups,
wherein the hydrogel material comprising a vinyl sulfone pendant group; and
(B) reacting the vinyl sulfone pendant groups with an quenching agent;
thereby forming the hydrogel.

57. The hydrogel product by the process of claim 56, wherein:
the crosslinking agent comprises divinyl sulfone, and
wherein the hydrogel comprises a residual vinyl sulfone concentration of at most about 2.0 μmol/mL of hydrogel.

58. A hydrogel composition comprising:
a hyaluronic acid network comprising a plurality of hyaluronic acid chains,
a sulfonyl bis-ethyl crosslink between hyaluronic acid chains,
a pendant group
a quenching agent wherein the quenching agent is covalently bonded to the pendent group.

59. (canceled)

60. A method of reducing toxicity of a divinyl sulfone crosslinked material comprising pendant vinyl groups, said method comprising the step of:
reacting the pendant vinyl sulfone group with a quenching agent
whereby the pendant vinyl sulfone group is converted to a less toxic material.

61. A method of reducing a number of pendant vinyl sulfone groups in a divinyl sulfone crosslinked material comprising pendant vinyl groups, said method comprising the step of: reacting the pendant vinyl sulfone group with a quenching agent whereby the number of pendant vinyl sulfone groups is reduced.

62. The method of claim 1, wherein the quenching agent comprises an amine, a polyamine, a polypeptide, a thiol, or an aminothiol.

63. The method of claim 1, wherein the quenching agent is lysine, glycine, cysteine, cysteamine, ethylenediamine, arginine, histidine, glutamate, aspartate, leucine, asparagine, methionine, serine, threonine, polyethylene amines, putrescine, spermine, thermospermine, spermidine, propane-1,3-dithiol, dimercaprol, ((aminopropyl)amino) ethanethiol, 3-(methylamino)-2-((methylamino)methyl) propane-1-thiol, or 3-amino-2-(aminomethyl) propane-1-thiol.
